(19) United States
(12) Patent Application Publication
(10) Pub. No.: US 2017/0168059 A1
(43) Pub. Date: Jun. 15, 2017

(54) BIOMARKERS FOR ASSESSING CANCER PATIENTS FOR TREATMENT

(71) Applicant: The Johns Hopkins University, Baltimore, MD (US)

(72) Inventors: Robert W. VELTRI, Baldwin, MD (US); Hui ZHANG, Ellicott City, MD (US); Christhunesa CHRISTUDASS, Tamilnadu (IN); Zhi LIU, Ellicott City, MD (US); Jonathan I. EPSTEIN, Baltimore, MD (US); H. Ballantine CARTER, Baltimore, MD (US); Guangjing ZHU, Columbia, MD (US); Christine DAVIS, Aberdeen, MD (US); Stephen M. HEWITT, Potomac, MD (US); Joon-Yong CHUNG, Rockville, MD (US)

(21) Appl. No.: 15/116,925

(22) PCT Filed: Feb. 9, 2015
(86) PCT No.: PCT/US15/15074

§ 371 Date: Aug. 5, 2016

Related U.S. Application Data
(60) Provisional application No. 61/937,075, filed on Feb. 7, 2014.

(51) Int. Cl. G01N 33/574

(52) U.S. Cl. CPC G01N 33/57434

(57) ABSTRACT

A method for selecting treatment options for patients is provided. The method comprises a procedure for selecting patients that should not be placed on Active Surveillance (AS) but receive active treatment even though the morphologic analysis of the patient's biopsy may show a Gleason score that would traditionally have placed the patient in AS without any further treatment. In accordance with one embodiment, the patient is selected for active treatment if a biomarker associated with prostate cancer is detected. In yet a further embodiment, the patient is selected for additional treatment if the biomarker's concentration is determined to be higher in the benign section of the tissue sample than in the cancerous section. In another embodiment, biochemical recurrence is predicted identifying patients for treatment.

### CROSS REFERENCE OF RELATED APPLICATIONS

This application claims the benefit of priority under 35 U.S.C. §119(e) from U.S. Provisional Ser. No. 61/937,075, filed 7 Feb. 2014, the entire contents of which are incorporated herein by reference.

### STATEMENT OF GOVERNMENT RIGHTS

This invention was made with government support under PC120857 and 5U01CA15283-03 awarded by Early Detection Research Network/NCI. The government has certain rights in the invention.

### FIELD

The disclosure relates to biomarkers and/or histology methods for the identification of early stage aggressive prostate cancer including assessing when a patient should be selected for curative intervention as compared to continued surveillance.

### BACKGROUND

A total of 40 men require treatment for prostate cancer (CaP) in order to save one man. Widespread prostate specific antigen (PSA) based screening is believed to have contributed to the decrease in prostate cancer mortality (1). A combination of opportunistic PSA screening and saturation extended pattern prostate biopsies has resulted in over-diagnosis and over-treatment up to ˜56% of the time in some men with low grade, low stage prostate cancer whose disease would have otherwise remained undetected during their lifetime (2-4). Such overtreatment can always create a chance of a decreased quality of life once sexual function and urinary function are compromised. While low grade low stage (indolent) CaP disease is over-detected and over-treated using traditional methods, such as PSA screening, lethal CaP is in contrast under-detected and under-treated.

There remains a need to accurately identify men with CaP that is destined (e.g., early stage aggressive) to progress to life threatening disease and who would benefit from curative intervention.

Since 1995, Johns Hopkins Medical Institutions (JHMI) has enrolled over 1100+ men in an active surveillance (AS) program with delayed surgical intervention as a treatment option. The JHMI AS program utilizes an IRB approved informed consent to prospectively obtain for all participants clinical and pathological specimens for research. Patients are enrolled if they meet Epstein inclusion very low risk criteria (5-10). AS patients are followed (i) semi-annually with serum total PSA (tPSA), free PSA (fPSA) and digital rectal examination, and (ii) annually with 12 core surveillance biopsy. (11). The AS program at JHMI is currently recommended for select individuals who have very low risk T1c (Cancer is found by needle biopsy that was done because of an increased PSA) disease and have less than a 20 year life expectancy, which is distinguished from other programs by their described selection criteria (11). This paradigm-shift stems from recent discoveries identifying important clinically and pathologically predictive pretreatment parameters that characterize very low to low risk disease based upon the extent of tumor burden (12-16). The National Comprehensive Cancer Network (NCCN) recognizes four separate risk groups of newly diagnosed patients with CaP (see Table 1 below) [17].

Current recognized risk categories are outlined in the Table 1 below [17].

| | Proportion of | |
|---|---|---|
| | newly | |
| Risk | Diagnosed | Management Recommendation from |
| Category | cases (%) | NCCN |
| Very Low* | 15 | Surveillance if life expectancy <20 yrs |
| | | (Test Subjects) |
| Low^{†} | 35 | Surveillance if life expectancy <10 yrs |
| | | Surveillance, radiation, or surgery if life |
| | | expectancy >10 yrs |
| Intermediate^{‡} | 40 | Radiation or surgery |
| High^{§} | 10 | Radiation or surgery |

*Very low risk (stage T1c, and PSA <10 ng/ml, and Gleason score ≦6, and no more than 2 cores containing cancer, and ≦50% of core involved with cancer, and PSA density <0.15)
^{†}Low risk (stage T1c or T2a, and PSA <10 ng/ml, and Gleason score ≦6)
^{‡}Intermediate risk (stage T2b, and PSA 10-20 ng/ml, and Gleason score 7)
^{§}High risk (stage >T2b, or PSA >20 ng/ml, or Gleason score ≧8)

However, a percentage of those patients categorized as “very low risk”, which are recommended for surveillance, instead have a disastrous outcome when a radical prostatectomy is performed and the patient actually is found to be in the “very high risk” category resulting in a serious and fatal outcome. Those patients classified by biopsy results to meet the Epstein criteria of “very low risk” but in fact contain indolent aggressive CaP, Intermediate or High risk, are referred to herein as Escapees. Hence, there is a need to further screen “Very low” and “Low” risk to identify Escapees to ensure proper consideration of treatment options.

Accordingly, provided herein are compositions and methods for identifying and predicting early clinically aggressive CaP in men that have indolent CaP.

### SUMMARY

The instant disclosure described methods for selecting treatment options for patients. In embodiments, the method provides a procedure for selecting patients with aggressive prostate cancer based upon their histologic Gleason scores and/or stages using retrospective archival samples configured in a tissue microarray. Also, we describe methods that prostate cancer patients should not be placed on active surveillance (AS) but receive active treatment even though the morphologic histologic analysis of the patient's biopsy may show a Gleason score that would traditionally have placed the patient in AS. In accordance with one embodiment, methods provided herein include identifying a patient for prostate cancer clinical intervention wherein the patient is currently identified for Active Surveillance of the cancer, comprising: i) measuring a level of at least one biomarker associated with aggressive prostate cancer in a sample from the patient wherein at least one of the biomarkers is CACNA1D; ii) calculating a probability of aggressive prostate cancer from said biomarker measurements; and, iii) identifying and selecting the patient for prostate cancer clinical intervention wherein the probability of aggressive prostate cancer indicates a likelihood that a patient has an early form of aggressive prostate cancer.

In further embodiments, methods comprise identifying more aggressive vs. less aggressive prostate cancer based upon radical prostatectomy specimens with long term follow-up that are configured as a tissue microarray (TMA) of which at least 2-3 molecular biomarkers (Ki67, CACNA1D, Her2/neu and/or Periostin), evaluated in the cancer or benign adjacent areas near the cancer.

In embodiments are provided methods for reducing the number of false negative cases associated with screening for prostate cancer, comprising: i) categorizing a patient based on measurement of circulating PSA and tissue morphology of a biopsy sample from the patient into low risk categories; ii) measuring a level of at least one biomarker associated with aggressive prostate cancer in the biopsy sample from those categorized as low risk wherein at least one of the biomarkers is CACA1ND; iii) calculating a probability of aggressive prostate cancer from said biomarker measurements; and, iv) re-categorizing a patient into an Intermediate or High Risk category for prostate cancer when the probability of aggressive prostate indicates a likelihood that a patient has an early form of aggressive prostate cancer, whereby the number of false negatives for prostate cancer is reduced. The low risk categories are defined by one of the following criteria, alone or in combination, i) PSA measurement of 10 ng/ml or less, ii) a Gleason score of 6 or less, iii) no more than 2 cores containing cancer in biopsy sample, iv) 50% or less of core involved with cancer in biopsy sample, and v) PSA density of less than 0.15. In one aspect, the low risk categories comprise Low risk and Very Low risk categories according to Table 1.

In embodiments, the re-categorized patient is selected for clinical intervention, wherein clinical intervention comprises surgery, chemotherapy, targeted drug therapeutic treatment (e.g. biologic therapeutic such as antibodies) or a combination thereof. In certain aspects the methods further comprising comparing presence of the biomarkers in a cancer section of the tissue sample and a benign section of the tissue sample, wherein the difference in the presence of the biomarker in the benign section of the tissue sample indicates the patient should be treated.

In certain embodiments, are provide methods for assessing the likelihood that a patient has an early form of aggressive prostate cancer, comprising: i) assessing tissue morphology of a biopsy sample from the patient including assigning a Gleason score; ii) measuring a level of prostate specific antigen (PSA) in a blood sample from the patient; iii) measuring a level of at least one biomarker associated with aggressive prostate cancer in the biopsy sample from the patient wherein at least one of the biomarkers is CACNA1D; and iv) calculating a probability of aggressive prostate cancer from said PSA measurement, biomarker measurements and tissue morphology of the biopsy sample, whereby the likelihood that a patient has an early form of aggressive prostate cancer is determined. In embodiments, the measured PSA 10 ng/ml or less, the Gleason score is 6 or less or a combination thereof. The methods may further comprising selecting those patients with a calculated probability for the likelihood of having an early form of aggressive prostate cancer for treatment.

### BRIEF DESCRIPTION OF THE DRAWINGS

The above and other features, aspects, and advantages of the present invention are considered in more detail, in relation to the following description of embodiments thereof shown in the accompanying drawings, in which:

FIG. 1 is a description of the specific antibodies used in the MTI protocol.

FIG. 2 is a picture of one example of the results of the multiplexing procedure utilizing six biomarkers.

FIG. 3 is a graphical representation and table of the Periostin biomarker.

FIG. 4 is a graphical representation and table of the CACNA1D biomarker.

FIG. 5 is a graphical representation and table of the HER2/neu biomarker.

FIG. 6 is a graphical representation and table of the HER2/neu biomarker for specific Gleason scores.

FIG. 7 is a graphical representation and table of the EZH2 biomarker.

FIG. 8 is a graphical representation and table of the (−7) ProPSA biomarker.

FIG. 9 is a graphical representation and table of the Ki67 biomarker.

FIG. 10 shows logistic regression graphs to assess biochemical recurrence (BCR) as a binary outcome using only Gleason score as a categorical variable to predict BCR.

FIG. 11 shows a stepwise multivariate logistic regression (MLR) graphs of biochemical recurrence (BCR) using Gleason grade+biomarkers as continuous variables to evaluate BCR: Pr=0.1.

FIG. 12 shows stepwise logistic regression graphs of Gleason score up to 7 vs. above 7 using biomarkers as continuous variable to evaluate Gleason score: Pr=0.1.

FIG. 13 shows stepwise logistic regression of Gleason score 3 and 4 (binary) using biomarkers as continuous variables to evaluate Gleason score outcome: Pr=0.1.

FIGS. 14 (A and B) shows a graphical representation and table of the Periostin biomarker comparing escapee and non-escapees.

FIGS. 15 (A and B) shows a graphical representation and table of the CACNA1D biomarker comparing escapee and non-escapees.

FIGS. 16 (A and B) shows a graphical representation and table of the HER2/neu biomarker comparing escapee and non-escapees.

FIGS. 17 (A and B) shows a graphical representation and table of the EZH2 biomarker comparing escapee and non-escapees.

FIGS. 18 (A and B) shows a graphical representation and table of the (−5,−7) proPSA biomarker comparing escapee and non-escapees.

FIG. 19 shows a graphical representation and table of the Periostin biomarker comparing cancer and benign area of the sample.

FIG. 20 shows a graphical representation and table of the CACNA1D biomarker comparing cancer and benign area of the sample.

FIG. 21 shows a graphical representation and table of the (−5,−7) proPSA biomarker comparing cancer and benign area of the sample.

FIG. 22 shows a graphical representation and table of the EZH2 biomarker comparing cancer and benign area of the sample.

FIG. 23 shows a graphical representation and table of the HER2/neu biomarker comparing cancer and benign area of the sample.

FIG. 24 shows a graphical representation and table of the HER2/neu biomarker comparing cancer and benign area of the sample and specific Gleason scores of 3+3 & 3+4 vs 4+3 and 8.

FIG. 25 shows a graphical representation and table of the Ki67 biomarker comparing cancer and benign area of the sample.

FIG. 26 is a table of the correlation analysis for the six biomarkers tested by MTI in the cancer area.

FIG. 27 is a graphical representation of the logistic regression to assess biochemical recurrence (BCR) as a binary outcome using only Gleason score as a categorical variable to predict BCR in the cancer area.

FIG. 28 shows a graphical representation of the stepwise multivariate logistics regression (MLR) of biochemical recurrence (BCR) using the six biomarkers as continuous variables to evaluate BCR: Pr=0.1 in the cancer area.

FIG. 29 shows a stepwise multivariate logistic regression (MLR) graph and table of biochemical recurrence (BCR) using Gleason grade+biomarkers BCR: Pr=0.1 in the cancer area.

FIG. 30 is a table of the correlation analysis for the six biomarkers tested by MTI in the benign area.

FIG. 31 is a graphical representation of the logistic regression to assess biochemical recurrence (BCR) as a binary outcome using only Gleason score as a categorical variable to predict BCR in the benign area.

FIG. 32 shows a graphical representation of the stepwise multivariate logistics regression (MLR) of biochemical recurrence (BCR) using the six biomarkers as continuous variables to evaluate BCR: Pr=0.1 in the benign area.

FIG. 33 shows a stepwise multivariate logistic regression (MLR) graph and table of biochemical recurrence (BCR) using Gleason grade+biomarkers BCR: Pr=0.1 in the benign area.

FIG. 34 shows a graphical representation of the six biomarkers comparing cancer and benign area of the sample with an AUC value of 0.98 (cancer) and 0.94 (cancer adjacent benign area).

FIG. 35 is a table of the BCR samples from the TMA 681 and 682.

FIG. 36 is a table of the demographics from the source of the 80 samples in the TMA 681 and 682.

FIG. 37 is a table of the model parameters (biomarkers in either cancer or benign areas) their odds ratios and p values.

FIG. 38 is a graphical representation for the prediction of BCR with Gleason score or the combination of Gleason score and the biomarkers CACNA1D and HER2/neu.

FIG. 39 is a graphical representation for the prediction of escapees status in the AS cohort with the biomarkers (−5,−7)proPSA and CACNA1D.

### DETAILED DESCRIPTION

### Introduction

The invention summarized above may be better understood by referring to the following description. This description of an embodiment, set out below to enable one to practice an implementation of the invention, is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

The present disclosure is based, in part, on the discovery of biomarkers associated with aggressive prostate cancer which can provide prognostic value, including i) identifying early stage aggressive prostate cancer incorrectly categorized as Low or Very Low risk and ii) predicting recurrence of prostate cancer. Provided herein are those individual biomarkers and panels thereof used in screening methods for determining the likelihood a patient has an early stage aggressive CaP, especially when categorized as Very Low or Low risk using traditional tissue morphology analysis and PSA measurement, and those patients in need of clinical intervention. Such intervention includes, but is not limited to, surgery, radiation, chemotherapy, etc.

Furthermore, those biomarkers can be used in combination with traditional categorization of prostate cancer, such as risk groups recognized by National Comprehensive Cancer Network (NCCN), to increase specificity and accuracy of correctly categorizing prostate cancer for appropriate treatment including predicting recurrence. Table 1, above, describes the risk group criteria recognized by NCCN, also referred to as Epstein criteria, for traditional classification and treatment recommendations for CaP.

In one embodiment, the accuracy of correctly categorizing the risk a patient has an aggressive form of prostate cancer is increased by reducing the number of false negatives (failures in Active Surveillance), i.e. those patients categorized as Very Low and Low risk but who in fact have an early form (e.g. indolent) of aggressive prostate cancer. The false negative rate, based on current screening methods recognized by the NCCN, is 25-30%.

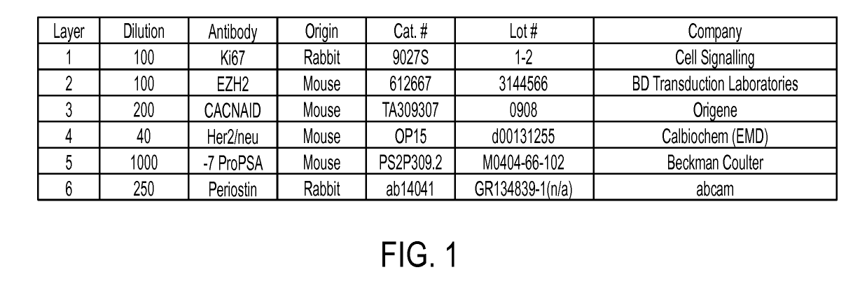

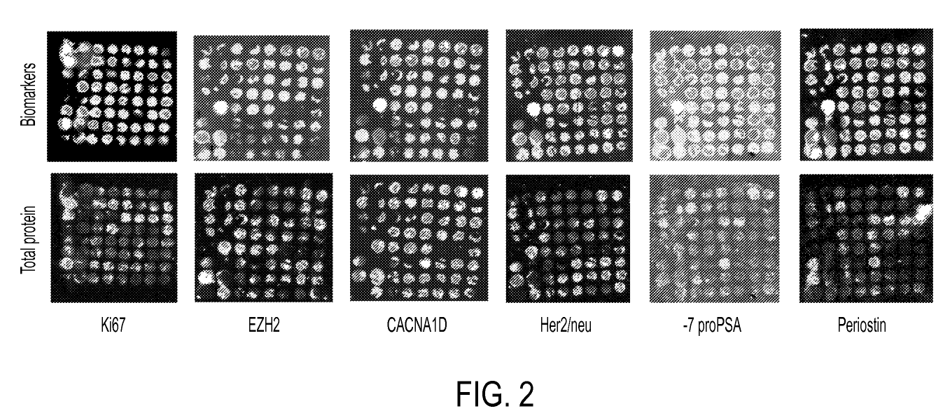

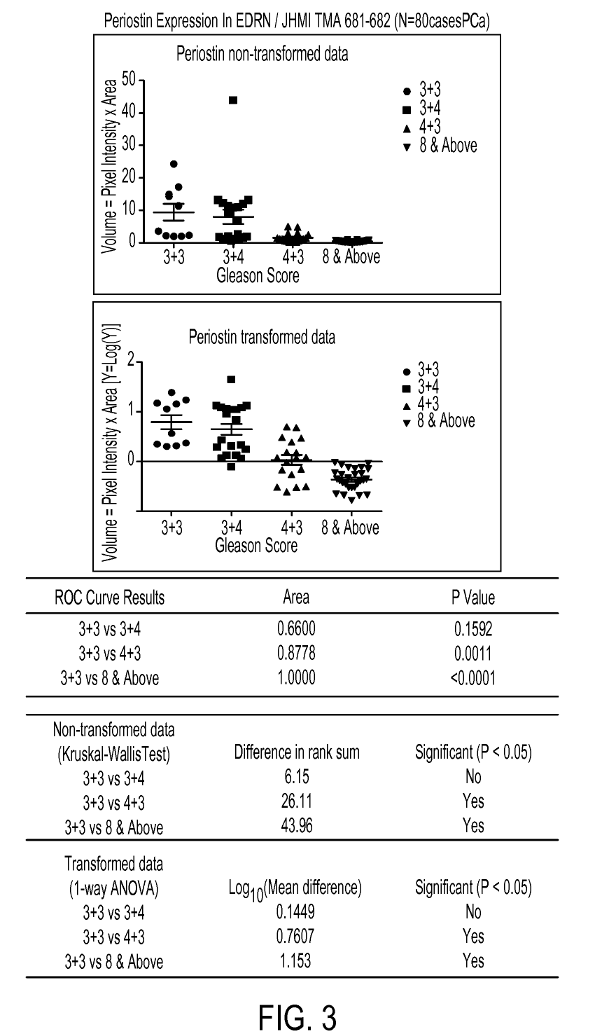

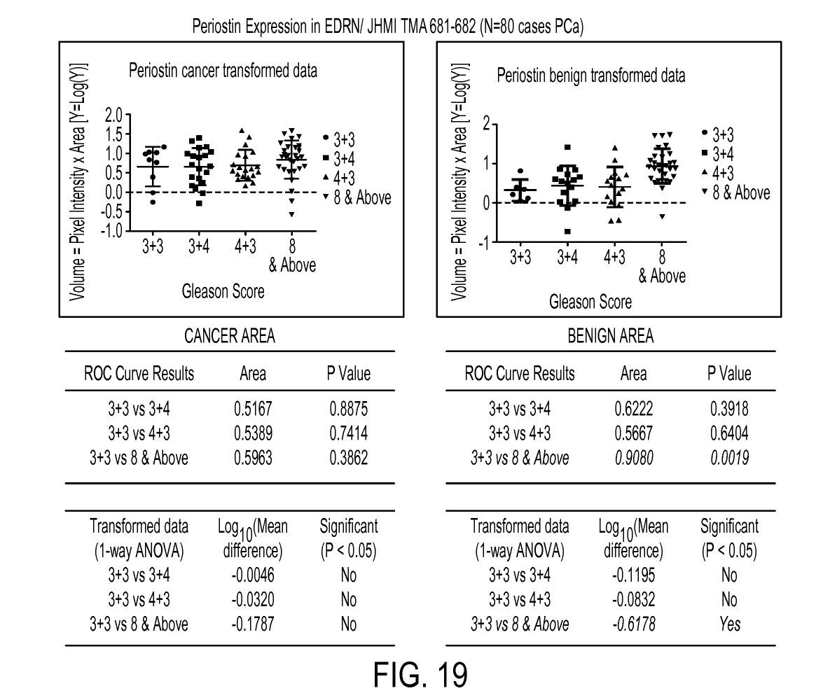

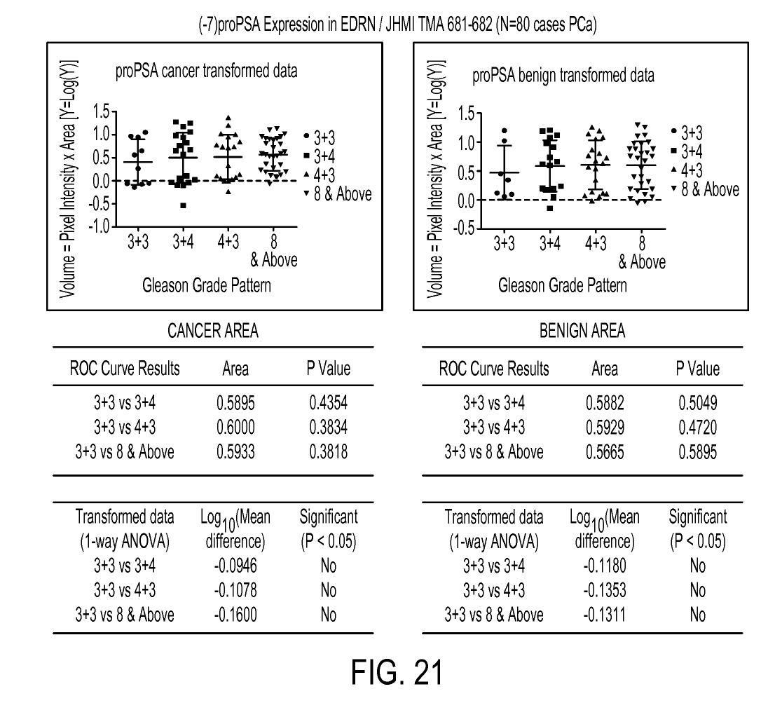

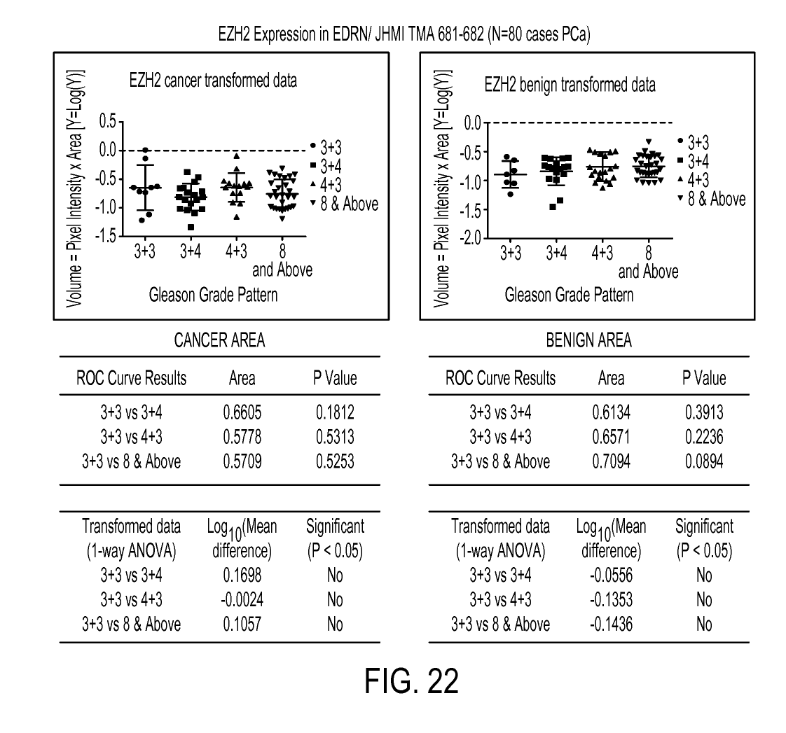

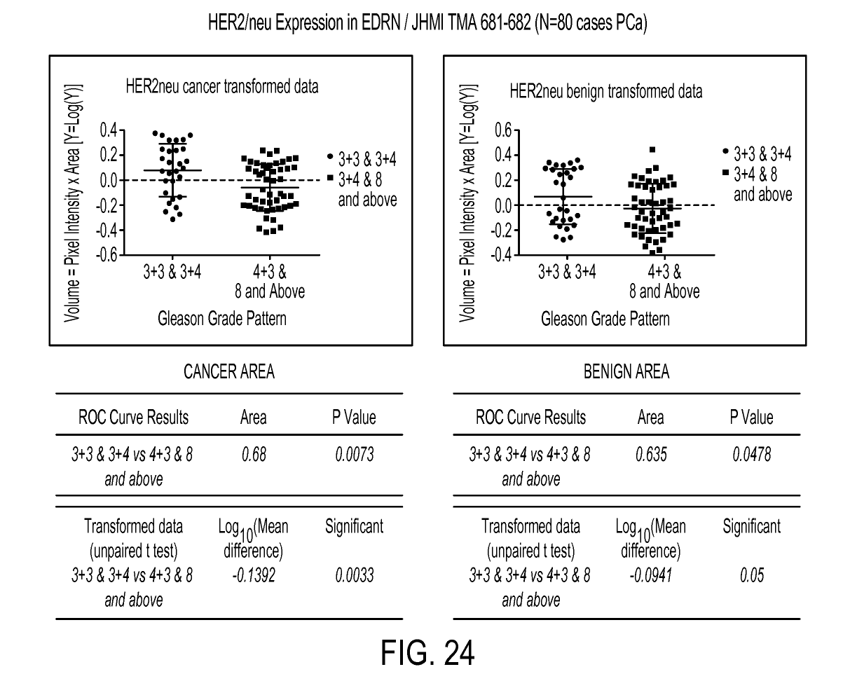

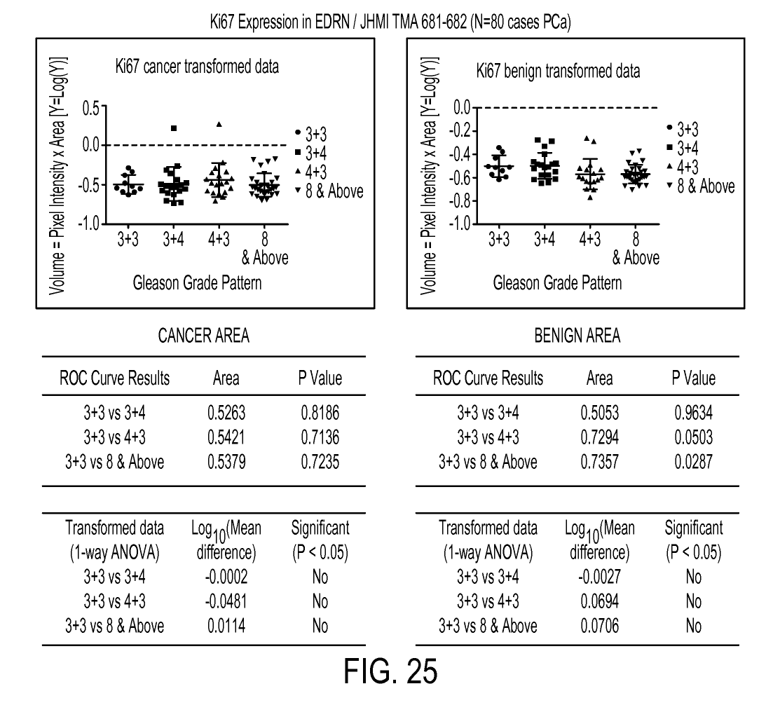

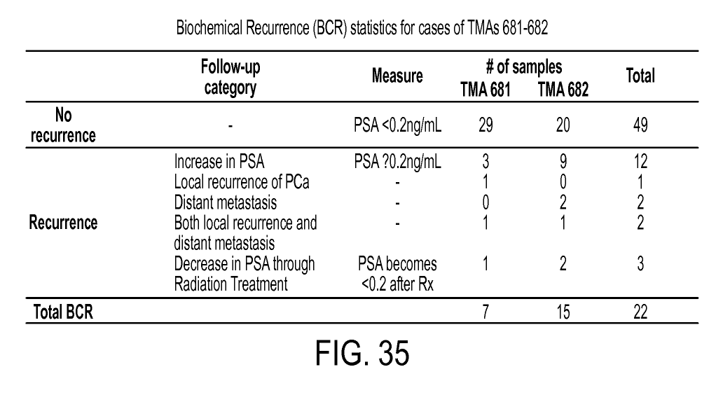

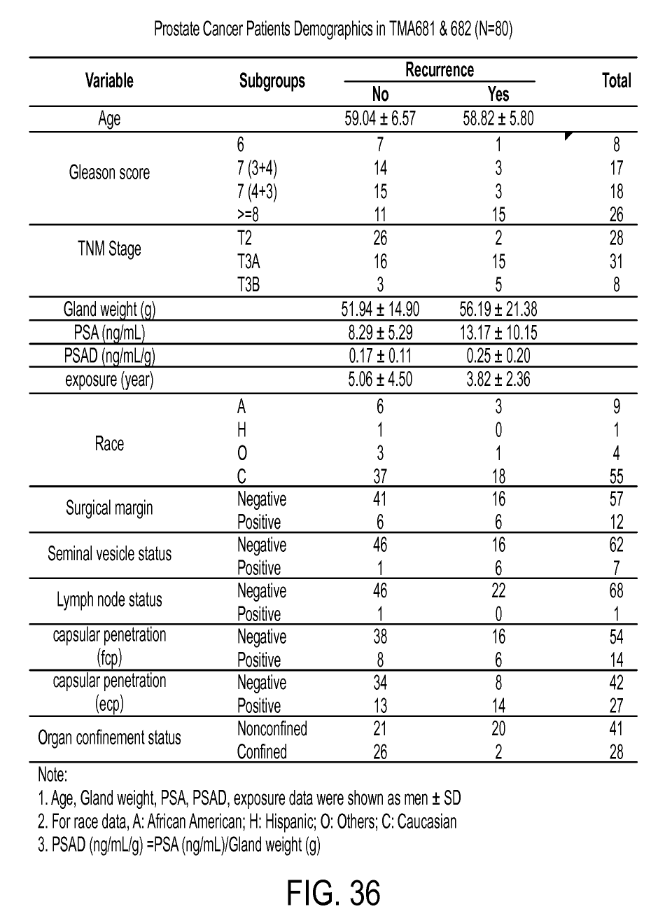

To test the prognostic value of the six identified biomarkers (See Example 2), samples from a cohort of 80 prostate cancer patients (See Example 3), with tumors graded as 3+3, 3+4, 4+3 and 8+, were screened using the protocol of Example 1 and Example 3 in both cancer areas and cancer adjacent benign areas. Two tissue microarrays (TMA 681 and 682) were constructed which included 22 cases of recurrence with a mean of ten years follow-up among the total of 80 cases. The recurrence cases comprise an increase in PSA, local recurrence of prostate cancer, distal metastasis, both local recurrence and distant metastasis, decrease in PSA through radiate treatment, see FIG. 35 for detailed information of the recurrence cases among the 80 cases in TMA 681 and 682 and FIG. 36 for detailed demographics of the total 80 cases separated by recurrence and further sub-grouped based on recurrence. See FIG. 1 for the specific antibodies used in the protocol of Example 1; FIG. 2 for a representative tissue microarray sample analysis; FIGS. 3-8 for individual analysis of each of the biomarkers; and, FIGS. 19-25 and 34 for a comparison of cancer and benign area analysis for each marker and as a panel of six biomarkers to distinguish indolent (Low and Very Low Risk category) from aggressive cancer (Intermediate and High Risk category).

Five of the six tested biomarkers (Periostin, CACNA1D, HER2/neu, EZH2 and (−5,−7)ProPSA) demonstrated an ability to distinguish between tumors categorized as Very Low or Low risk (3+3 grade) and High risk (8+ grade). Four of the tested biomarkers (Periostin, CACNA1D, EZH2 and (−5,−7)ProPSA) also demonstrated an ability to identify and distinguish those tumors categorized as intermediate (4+3 grade) from Low (3+3 grade) and High (8+ grade) risk categorized CaP. The panel of six biomarkers demonstrated and an ability to distinguish between indolent and aggressive cancer with a sensitivity of 95% and specificity of 95.8% in cancer areas.

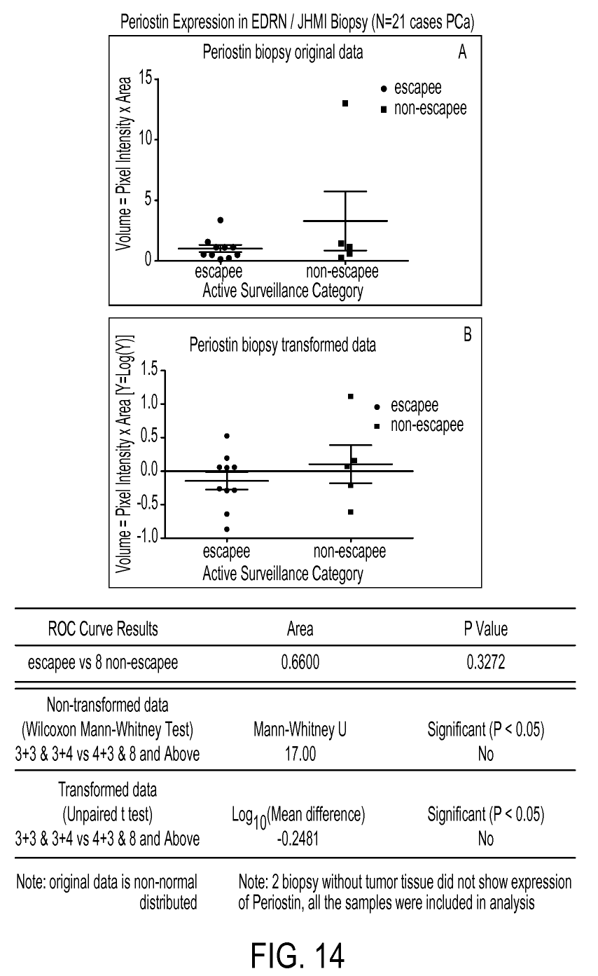

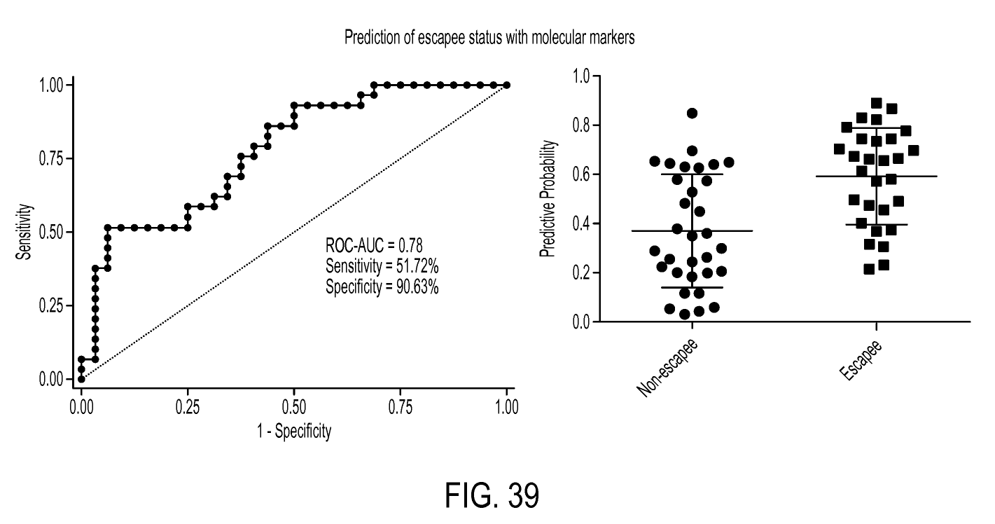

To test the efficacy, e.g ability to differentiate indolent from aggressive prostate cancer, of the biomarkers in identifying subjects classified as Very Low or Low risk using traditional tissue analysis and PSA measurement per NCCN guidelines, but who in fact have undiagnosed early stage aggressive prostate cancer “Escapees”, a cohort of 21 Active Surveillance Escapees and Controls were screened using the six biomarkers. See Example 4 and FIGS. 14-18 (n=21) and 39 (n=29). Of the biomarkers tested, CACNAID alone, or the combination of (−5,−7)ProPSA and CACNAID, were able to correctly identify Escapees with statistical significance, see FIG. 39.

The biomarkers were further analyzed to identify patients for clinical intervention based on differential concentration between cancer and non-cancerous (benign cancer adjacent) portions of the tissue sample. See FIGS. 19-25, 37 and Example 5. The data demonstrates the differential expression of the measured biomarkers (e.g., Periostin, CACNA1D and Her2/neu) can be used for determining the likelihood a patient will progress to aggressive CaP and require additional early clinical intervention.

To explore the difference of cancer area and the benign adjacent area for predicting recurrence, the 6 biomarkers, Gleason score and recurrence in both areas was analyzed in the 80 samples from the TMA 681 and 682. See, Example 1 and 3. The results showed that CACNA1D, an epithelial expressed Ca2+ transporter glycoprotein biomarker, demonstrated a strong and moderate negative correlation with Gleason score in cancer and benign areas, respectively; while Her2/neu showed a moderate correlation in both cancer and benign areas. The other markers were weakly correlated with Gleason score.

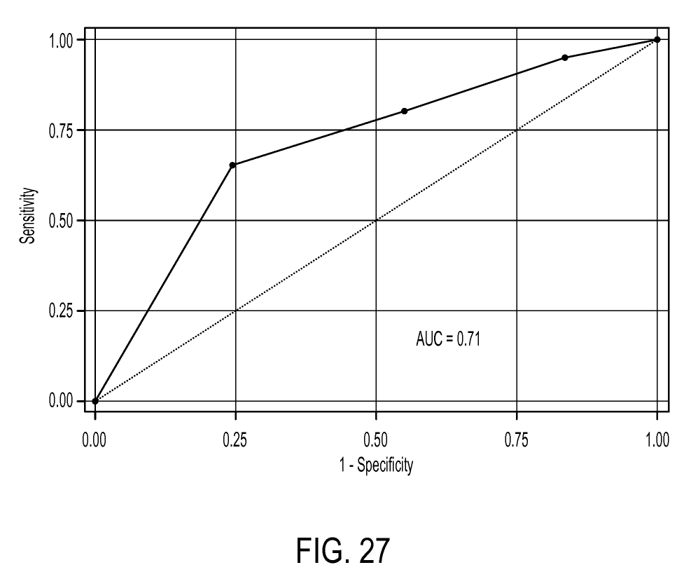

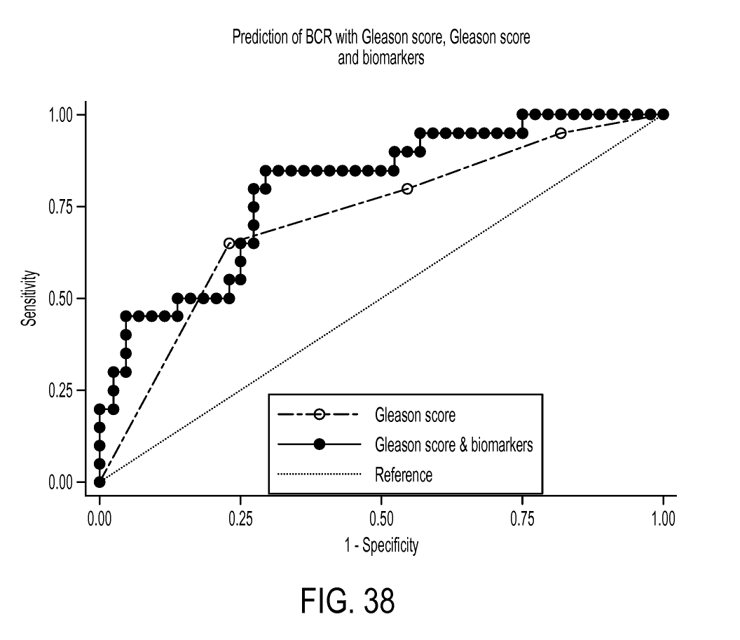

In certain embodiments, the combination of Gleason score, Her2/neu and CACNA1D may be used to predict the recurrence of CaP, wherein the AUC (Area Under the Curve) value as determined by a ROC (Receiver Operating Characteristic) analysis was 0.79. See FIG. 38. Interestingly, in the benign adjacent area, the combination of Gleason score and Her2/neu could be used to predict recurrence with an AUC value of 0.73 while (−5,−7)ProPSA also showed weaker prediction with an AUC of 0.67. Therefore, combination of different cancer biomarkers in MTI may be used to predict the recurrence of PCa. See FIGS. 27-33

In embodiments are provided a screening method for identifying individuals with an early stage aggressive prostate cancer (CaP) utilizing biomarkers associated with aggressive prostate cancer. In one aspect, the individual was categorized as being in a low risk group for developing aggressive CaP. Such categorization of risk is typically determined by a combination of measured PSA and tissue histomorphology, including assigning a Gleason score or Gleason Grade. In certain aspects, biopsy samples are first subjected to histomorphometric analysis, such as determining microscopic tumor grade patterns and volumes, and based on the sample's Gleason score, e.g. ≦6, the sample is then selected for biomarker screening using biomarkers shown to differentiate indolent prostate cancer from an aggressive form and/or biomarkers associated with aggressive CaP. In another aspect, the patient is screened with a biomarker, or panel of biomarkers associated with aggressive CaP, without first being categorized according to Table 1. The presence of specific biomarkers in biopsy samples are measured, both qualitatively or quantitatively depending on the screening methods and subsequent analysis. Ultimately, the approach will select patient's samples that show a minimum threshold or cut-off of a specific biomarker, or combination of biomarkers, that are then selected for clinical intervention. In other words, the present methods select those patients for active treatment that would otherwise be managed by surveillance methods, but who have an early form of aggressive prostate cancer by calculating the probability the patient has an early form of aggressive prostate cancer based on the biomarker measurement and/or PSA measurement and/or tissue morphology of a biopsy sample from the patient. The methods also provide a means for screening asymptomatic subjects and correctly categorizing them as Very Low, Low, Intermediate or High risk for developing aggressive CaP without the need for PSA measurement and histology analysis.

In embodiments, the present methods provide for testing patients when they would previously have been placed on active surveillance (AS) without further testing based on at least the morphological analysis of a biopsy tissue sample. There are a certain number of patients who are categorized as Very Low or Low risk who nonetheless have an early, and until now, undetected form of aggressive prostate cancer; herein referred to as “Escapees”. Surprisingly, it was possible to detect biomarkers (e.g. CACNA1D) associated with aggressive prostate cancer in a certain group of patients that were determined to have an early stage of aggressive CaP. Typically, as described in the prior art, patients would have been placed on active surveillance without further testing if at least their Gleason scores were <6, and/or in combination with <2 cores containing cancer, and <50% per core involved with cancer, e.g. those patients categorized as Very Low or Low risk. In such instances, it would not have been expected that these biomarkers would be present in sufficient quantity for detection.

In another embodiment is provided an algorithm for the identification of patients at risk of developing aggressive CaP or those with an early stage of aggressive CaP. The algorithm utilizes the correlations between various markers for CaP and their presence in tissue samples when the patient's biopsy shows a Gleason scores <6, <2 cores containing cancer, and <50% per core involved with cancer. The algorithm can be implemented through a computer program. The computer program can be designed to determine the statistical probability of a particular patient developing aggressive CaP based on the intensity of a particular biomarker on a tissue sample membrane (described in more detail below). See Example 1 and 4.

In embodiments provided herein are methods where the benign area of the test is used in order to determine propensity to develop aggressive CaP. In this particular embodiment, the quantity of a specific biomarker is measured in both the benign (non-cancerous) and malicious (cancerous) section of a tissue sample. Surprisingly, the biomarker's intensity (e.g. Periostin) in the benign area of the tissue sample increases in relation to a heightened Gleason score, while the intensity in the malicious section remains constant or decreases.

In embodiments, are provided methods comprises comparing the intensity of a biomarker in a cancerous and non-cancerous section of a tissue sample. A change of intensity in the specific biomarker in the benign area indicates that the patient is likely to develop aggressive CaP and additional therapy options beyond AS need to be presented to the patient.

### DEFINITIONS

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

As used herein, the term “active surveillance”, “objective surveillance” or “watchful waiting”, used interchangeably herein, refer to active observation and regular monitoring of a patient without actual treatment. Patients categorized as Very Low and Low risk are recommended for surveillance depending on their life expectancy. See Table 1.

As used herein, the term “AUC” refers to the Area Under the Curve, for example, of a ROC Curve. That value can assess the merit of a test on a given sample population with a value of 1 representing a good test ranging down to 0.5 which means the test is providing a random response in classifying test subjects. Since the range of the AUC is only 0.5 to 1.0, a small change in AUC has greater significance than a similar change in a metric that ranges for 0 to 1 or 0 to 100%. When the % change in the AUC is given, it will be calculated based on the fact that the full range of the metric is 0.5 to 1.0. A variety of statistics packages can calculate AUC for an ROC curve, such as, SigmaPlot 12.5, JMP™, STATA™ or Analyse-It™. AUC can be used to compare the accuracy of the classification algorithm across the complete data range. Classification algorithms with greater AUC have, by definition, a greater capacity to classify unknowns correctly between the two groups of interest (disease and no disease). The classification algorithm maybe as simple as the measure of a single molecule or as complex as the measure and integration of multiple molecules.

As used herein, the term “CaP biomarkers” refers to prostate cancer biomarkers associated with aggressive CaP and can accurately identify those tumors classified as High risk for developing aggressive CaP. In embodiments, the CaP biomarkers can also accurately identify those tumors classified as Intermediate risk for developing aggressing CaP. In certain embodiments the CaP biomarkers are capable of distinguishing between indolent prostate tumors and lethal or aggressive prostate tumors.

As used herein, the terms “differentially expressed gene,” “differential gene expression” and their synonyms, which are used interchangeably, are used in the broadest sense and refers to a gene and/or resulting protein whose expression is activated to a higher or lower level in a subject suffering from a disease, specifically cancer, such as lung cancer, relative to its expression in a normal or control subject. The terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products (e.g. proteins), or a comparison of the ratios of the expression between two or more genes or their gene products, or even a comparison of two differently processed products of the same gene, which differ between normal subjects and subjects suffering from a disease, specifically cancer, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages.

As used herein, the term “Escapee” refers to a patient sample that was assigned to active surveillance (AS) group based on the scoring and risk categories of Table 1 (e.g. Very Low or Low risk), but later exhibited early aggressive prostate cancer (CaP) and as used herein the term “non-Escapee” refers to a patient that was categorized as Very Low or Low and did not develop aggressive CaP

As used herein, the term “gene expression profiling” is used in the broadest sense, and includes methods of quantification of mRNA and/or protein levels in a biological sample.

As used herein, the term “Gleason Grading System” is a system of grading prostate cancer. The Gleason grading system assigns a grade that indicates the degree of differentiation of the tumor. Grades range from 1 to 5, with 1 being the most well differentiated and least aggressive and 5 being the most poorly differentiated and the most aggressive. Grade 3 tumors, for example, seldom have metastases, but metastases are common with grade 4 or grade 5. The first and second most abundant Gleason areas of the tumor are then added together to produce a Gleason score. A score of 2 to 4 is considered low grade; 5 through 7, intermediate grade; and 8 through 10, high grade. A tumor with a low Gleason score typically grows slowly enough that it may not pose a significant threat to the patient in his lifetime.

The Gleason scoring system is based on microscopic patterns of differentiation of the tumor as assessed by a pathologist while interpreting the biopsy specimen. When prostate cancer is present in the biopsy, the Gleason score is based upon the degree of loss of the normal glandular tissue architecture (i.e. shape, size and lumen formation of the glands) as originally described and developed by Dr. Donald Gleason in 1977. The classic Gleason scoring diagram shows five basic tissue patterns that are technically referred to as tumor “grades”. The subjective microscopic determination of this loss of normal glandular structure caused by the cancer is abstractly represented by a grade, a number ranging from 1 to 5, with 5 being the worst grade possible. The Gleason score (GS) and the Gleason sum are one and the same. However, the Gleason grade and the Gleason score or sum are different. The biopsy Gleason score is a sum of the primary grade (representing the majority of tumor) and a secondary grade (assigned to the minority of the tumor), and is a number ranging from 2 to 10. Today, the Gleason score tends to routinely be interpreted as 5-10. The higher the Gleason score, the more aggressive the tumor is likely to act and the worse the patient's prognosis.

As used herein, the term “level” of one or more biomarkers refers to the absolute or relative amount or concentration of the biomarker in the sample.

As used herein “Managing subject treatment” refers to the behavior of the clinician or physician subsequent to the determination of prostate cancer status. For example, if the result of the methods of the present invention is inconclusive or there is reason that confirmation of status is necessary, the physician may order more tests. Alternatively, if the status indicates that surgery is appropriate, the physician may schedule the patient for surgery. Likewise, if the status is negative, e.g., late stage prostate cancer or if the status is acute, no further action may be warranted. Furthermore, if the results show that treatment has been successful, no further management may be necessary. “Clinical Intervention”, as used herein, refers to any of these measures taken by the physician that is more than active surveillance, e.g. surgery, radiation, chemotherapy, etc.

As used herein, the terms “marker”, “biomarker” (or fragment thereof) and their synonyms, which are used interchangeably, refer to molecules that can be evaluated in a sample and are associated with a physical condition. For example, a marker includes tissue architecture, cellular or nuclear morphological alterations, expressed genes or their products (e.g. proteins) or autoantibodies to those proteins that can be detected from a human samples, such as blood, serum, solid tissue, and the like, that, that is associated with a physical or disease condition, or any combination thereof. Such biomarkers include, but are not limited to, biomolecules comprising nucleotides, amino acids, sugars, fatty acids, steroids, metabolites, polypeptides, proteins (such as, but not limited to, antigens and antibodies), carbohydrates, lipids, hormones, antibodies, regions of interest which serve as surrogates for biological molecules, combinations thereof (e.g., glycoproteins, ribonucleoproteins, lipoproteins) and any complexes involving any such biomolecules, such as, but not limited to, a complex formed between an antigen and an autoantibody that binds to an available epitope on said antigen. The term “biomarker” can also refer to a portion of a polypeptide (parent) sequence that comprises at least 5 consecutive amino acid residues, preferably at least 10 consecutive amino acid residues, more preferably at least 15 consecutive amino acid residues, and retains a biological activity and/or some functional characteristics of the parent polypeptide, e.g. antigenicity or structural domain characteristics. It is also understood in the present methods that use of the markers in a panel may each contribute equally to the composite score or certain biomarkers may be weighted wherein the markers in a panel contribute a different weight or amount to the final composite score.

As used herein the term “normalization”, refers to conversion of the quantitative numerical values into numerical values which can be compared with gene expression amounts obtained by other gene expression analyses. Typically, normalization is carried out by using the gene expression levels of a housekeeping gene being steadily expressed is used as an index for normalization of gene expression amounts.

As used herein, the term “a positive predictive score,” “a positive predictive value,” or “PPV” refers to the likelihood that a score within a certain range on a biomarker test is a true positive result. This is also referred to herein as a probability of cancer, represented as a percentage. It is defined as the number of true positive results divided by the number of total positive results. True positive results can be calculated by multiplying the test Sensitivity times the Prevalence of disease in the test population. False positives can be calculated by multiplying (1 minus the Specificity) times (1−the prevalence of disease in the test population). Total positive results equal True Positives plus False Positives.

As used herein, the term “probability of cancer”, refers to a probability or likelihood (e.g. represented as a percentage) that a patient, after screening using the present methods, is positive for the presence of early stage aggressive CaP.

As used herein the term “Prostate cancer” refers to a disease in which cancer develops in the prostate, a gland in the male reproductive system. “Low grade” or “lower grade” prostate cancer refers to non-metastatic prostate cancer, including malignant tumors with low potential for metastasis (i.e. prostate cancer that is considered to be “less aggressive”). Cancer tumors that are confined to the prostate (i.e. organ-confined, OC) are considered to be less aggressive prostate cancer. “High grade” or “higher grade” prostate cancer refers to prostate cancer that has metastasized in a subject, including malignant tumors with high potential for metastasis (prostate cancer that is considered to be “aggressive”). Cancer tumors that are not confined to the prostate (i.e. non-organ-confined, NOC) are considered to be aggressive prostate cancer. Tumors that are confined to the prostate (i.e., organ confined tumors) are considered to be less aggressive than tumors which are not confined to the prostate (i.e., non-organ confined tumors). “Aggressive” prostate cancer progresses, recurs and/or is the cause of death. Aggressive cancer may be characterized by one or more of the following: non-organ confined (NOC), association with extra capsular extensions (ECE), association with seminal vesicle invasion (SVI), association with lymph node invasion (LN), association with a Gleason Score major or Gleason Score minor of 4, and/or association with a Gleason Score Sum of 8 or higher. In contrast “less aggressive” cancer is confined to the prostate (organ confined, OC) and is not associated with extra capsular extensions (ECE), seminal vesicle invasion (SVI), lymph node invasion (LN), a Gleason Score major or Gleason Score minor of 4, or a Gleason Score Sum of 8 or higher. See Table 1 for recognized risk categories of prostate cancer.

As used herein the term, “Receiver Operating Characteristic Curve,” or, “ROC curve,” is a plot of the performance of a particular feature for distinguishing two populations, patients with lung cancer, and controls, e.g., those without lung cancer. Data across the entire population (namely, the patients and controls) are sorted in ascending order based on the value of a single feature. Then, for each value for that feature, the true positive and false positive rates for the data are determined. The true positive rate is determined by counting the number of cases above the value for that feature under consideration and then dividing by the total number of patients. The false positive rate is determined by counting the number of controls above the value for that feature under consideration and then dividing by the total number of controls.

ROC curves can be generated for a single feature as well as for other single outputs, for example, a combination of two or more features that are combined (such as, added, subtracted, multiplied etc.) to provide a single combined value which can be plotted in a ROC curve.

The ROC curve is a plot of the true positive rate (sensitivity) of a test against the false positive rate (1−specificity) of the test. ROC curves provide another means to quickly screen a data set.

As used herein, the term “sample” or “biological sample” refers to a biological material isolated from a subject such as a biopsy specimen. The biological sample may contain any biological material suitable for detecting the desired biomarkers, and may comprise cellular and/or non-cellular material from the subject. The sample can be isolated from any suitable biological tissue or fluid such as, for example, prostate tissue, blood, blood plasma, urine, or cerebral spinal fluid (CSF).

As used herein, the term “screening” refers to a strategy used in a population to identify an unrecognized cancer in asymptomatic or low risk subjects, for example those without signs or symptoms of the cancer. As used herein, a cohort of the population (e.g. Very Low or Low risk) are screened for a particular cancer (e.g. aggressive CaP) wherein the present methods are applied to determine the likelihood and/or risk to those Low subjects for the presence of the cancer.

As used herein, the term “sensitivity” refers to statistical analysis that measures the proportion of positives which are correctly identified as positives: true positives. The higher the sensitivity the fewer false negatives are identified. The sensitivity, at a designated specificity cutoff (e.g., 80%), of a biomarker or panels or biomarkers for a particular disease (e.g., aggressive CaP) can be measured and used to assess a patient's risk for the particular disease.

As used herein, the term “specificity” refers to statistical analysis that measures the proportion of negatives which are correctly identified as negative; true negatives. The higher the specificity the lower the false positive rate. The higher the combined specificity (e.g., 80%) and sensitivity (e.g., at least 80%) the better predictor a biomarker, or panel of biomarkers, are for correctly identifying aggressive CaP with clinical utility.

As used herein, the term “tumor,” refers to all neoplastic cell growth and proliferation, whether malignant or benign, and all pre-cancerous and cancerous cells and tissues.

### Biomarkers

The present disclosure is directed to one more aggressive CaP biomarkers and their use in screening for forms of early stage aggressive CaP or patients with a likelihood of developing aggressive CaP or for predicting recurrence in patients of prostate cancer. As used herein “screening for aggressive CaP” refers to diagnosing aggressive prostate cancer in a patient and/or determining the likelihood of aggressive prostate cancer in a patient and/or categorizing a patient's risk for aggressive prostate cancer and/or determining a patient's increased risk for aggressive prostate cancer and/or for predicting recurrence of aggressive prostate cancer. In certain embodiments, patients categorized as Very Low or Low risk are screened using the present biomarkers (NCCN, 2010). In other embodiments, asymptomatic patients are screened using the present biomarkers.

In certain embodiments are provided biomarkers, one or a panel of biomarkers, that can distinguish indolent prostate cancer (e.g. inactive or relatively benign) from early stage aggressive CaP. In another embodiment are provided biomarkers, one or a panel of biomarkers, that can identify those patients with a likelihood to develop aggressive CaP. In another embodiment, are provided biomarkers, one or a panel of biomarkers, that can correctly identify patients as High risk for developing aggressive CaP. In yet another embodiment, are provided biomarkers, one or a panel of biomarkers, that can correctly identify patients as Intermediate risk for developing aggressive CaP. In another embodiments, are provided biomarkers, one or a panel of biomarkers, that can predict recurrence of aggressive prostate cancer, either alone or in combination with a Gleason score.

In certain aspects, the biomarker panel comprises at least one, at least two, at least three, at least four, at least five, at least six, at least seven, at least eight, at least nine, at least 10, at least 15, at least 20, at least 30, at least 40 or at least 50 aggressive prostate cancer biomarkers.

It is understood that for any of the aggressive prostate cancer biomarkers or panels described herein, the panel measures the biomarker listed in the panel and that the panel does not comprise that biomarker but rather the means to measure the level in a sample of that stated biomarker.

However, before measurement can be performed, biomarkers need to be selected for screening aggressive prostate cancer. Many biomarkers are known for prostate cancer and a panel can be selected, or as was done by the present Applicants, biomarkers can be selected based on measurement of individual markers in retrospective clinical samples wherein a list of biomarkers is generated based on empirical data that are associated (strong and/or moderate correlation) with aggressive prostate cancer.

That biomarker, one or a panel, may be trained using a training set and validation set, to differentiate indolent CaP from early stage aggressive CaP.

Examples of biomarkers that can be employed include measurable molecules, for example, in a body fluid sample, such as, antibodies, antigens, small molecules, proteins, hormones, genes and so on, wherein the present aggressive prostate cancer biomarkers comprises at biomarkers that can identify those patients with a likelihood to develop aggressive CaP and/or biomarkers capable of distinguishing indolent CaP from early stage aggressive CaP.

In one exemplary embodiment, any one of nine (9) biomarkers (Periostin, CACNA1D, EZH2, Her2/neu, (−5,−7) ProPSA, P300, Ki67, RBM3, and PBOV-1) can be utilized to characterize an aggressive CaP phenotype. In certain embodiments, the biomarkers are used as a panel to identify and distinguish Intermediate and/or High risk CaP tumors for developing aggressive CaP. In certain other embodiments, the biomarker is CACNA1D and is used to distinguish indolent tumors from aggressive CaP tumors. The biomarkers can be measured in biopsy samples, both those of newly prepared tissue and archival tissue previously analyzed using histology methods.

### Methods of Screening for Early Stage Aggressive CaP Cancer Using the Prostate Cancer Biomarkers

In embodiments, provided herein are methods for screening a patient for early stage aggressive prostate cancer. Screening, includes, but is not limited to using the present prostate cancer biomarkers for diagnosing aggressive prostate cancer in a patient and/or determining the likelihood of early stage aggressive prostate cancer in a patient and/or re-categorizing a patient's risk for aggressive prostate cancer and/or determining a patient's increased risk for aggressive prostate cancer and/or reducing the false negatives in the Active Surveillance group (e.g. failures). In one aspect, the risk level is increased as compared to the Very Low or Low risk groups. In another aspect, the risk level of the patient is re-categorized from Very Low or Low to Intermediate or High risk. The Very Low or Low risk patients that, after testing, have a quantified increased risk for the presence of early stage aggressive prostate cancer or a likelihood of developing aggressive CaP are those that a physician may recommend for clinical intervention.

Therefore, in embodiments, are provided methods for assessing the likelihood that a patient has early stage aggressive prostate cancer, comprising 1) measuring a level of at least one prostate cancer biomarker in a sample from the human subject, wherein the biomarker is associated with aggressive CaP; 2) calculating a probability of early stage aggressive CaP from said biomarker measurements, whereby the likelihood that a patient will develop aggressive CaP is determined. In one aspect, a probability of early stage aggressive CaP means a patient is re-categorized as Intermediate or High risk for developing aggressive CaP.

In other embodiments, are methods for assessing the likelihood that a patient has early stage aggressive prostate cancer, comprising 1) selecting those patients that are Very Low or Low risk for aggressive CaP; 2) measuring a level of at least one prostate cancer biomarker in a sample from the human subject, wherein the biomarker is capable of distinguishing indolent from aggressive CaP; 3) calculating a probability of early stage aggressive CaP from said biomarker measurements, whereby the likelihood that a patient will develop aggressive CaP is determined. In one aspect, the biomarker is CACNA1D.

In certain other embodiments, are methods for selecting patients for clinical intervention of prostate cancer, comprising 1) selecting those patients that are Very Low or Low risk for developing aggressive CaP; 2) measuring a level of at least one prostate cancer biomarker in a sample from the human subject, wherein the biomarker is capable of distinguishing indolent from aggressive CaP; 3) calculating a probability of early stage aggressive CaP from said biomarker measurements; 4) selecting the individual for clinical intervention that is re-categorized as Intermediate or High risk for developing aggressive CaP from the biomarker measurements. In one aspect, the biomarker is CACNA1D.

In certain other embodiments, are methods for predicting biochemical recurrence associated with prostate cancer, comprising; i) obtaining a tissue biopsy sample from a patient; ii) assessing tissue morphology of a biopsy sample from the patient including assigning a Gleason score; iii) measuring a level of at least one biomarker associated with aggressive prostate cancer in the biopsy sample iv) calculating a probability of recurrence of aggressive prostate cancer from said biomarker measurements and Gleason score of the biopsy sample, whereby the biochemical recurrence is predicted.

One or more steps of the method described herein can be performed manually or can be completely or partially automated (for example, one or more steps of the method can be performed by a computer program or algorithm. If the method were to be performed via computer program or algorithm, then the performance of the method would further necessitate the use of the appropriate hardware, such as input, memory, processing, display and output devices, etc.). Methods for automating one or more steps of the method would be well within the skill of those in the art.

The methods described herein allowed Applicants to predict those men that would have required immediate treatment based on biopsy classifiers that use morphologic and biomarkers. The computational solution includes clinical, quantitative tissue morphologic biomarkers and pre-selected protein and Glycoprotein biomarkers developed tissue assays using novel tissue multiplexing immunohistochemistry (IHC) technology.

### Measuring Biomarkers in a Sample

The first step in the present method is measuring at least one biomarker, following sample collection, from a human subject, such as a patient categorized as Very Low or Low risk for developing aggressive CaP. There are many methods known in the art for measuring gene expression (e.g. mRNA) or the resulting gene products (e.g. polypeptides or proteins) that can be used in the present methods. The sample typically includes biopsy tissue, but may also include blood, plasma, spinal fluid or urine and is processed so that CaP biomarkers are measured from the sample. In certain embodiments, the sample is from a patient suspected of having early stage aggressive CaP or at risk of developing aggressive CaP. In other embodiments, the patient is categorized as Very Low or Low risk for developing aggressive CaP. In one aspect, the patient is asymptomatic for CaP.

The presence and quantification of one or more biomarkers, e.g. protein or peptides, in a test sample can be determined using one or more immunoassays that are known in the art. Immunoassays typically comprise: (a) providing an antibody (or antigen) that specifically binds to the biomarker (namely, protein or peptide); (b) contacting a test sample with the antibody or antigen; and (c) detecting the presence of a complex of the antibody bound to the antigen in the test sample or a complex of the antigen bound to the antibody in the test sample.

Well known immunological binding assays include, for example, an enzyme linked immunosorbent assay (ELISA), which is also known as a “sandwich assay”, an enzyme immunoassay (EIA), a radioimmunoassay (RIA), a fluoroimmunoassay (FIA), a chemiluminescent immunoassay (CLIA) a counting immunoassay (CIA), a filter media enzyme immunoassay (MEIA), a fluorescence-linked immunosorbent assay (FLISA), agglutination immunoassays and multiplex fluorescent immunoassays (such as the Luminex Lab MAP), immunohistochemistry, etc. For a review of the general immunoassays, see also, Methods in Cell Biology: Antibodies in Cell Biology, volume 37 (Asai, ed. 1993); Basic and Clinical Immunology (Daniel P. Stites; 1991).

The immunoassay can be used to determine a test amount of the biomarker in a sample from a subject. First, a test amount of an antigen in a sample can be detected using the immunoassay methods described above. If an antigen is present in the sample, it will form an antibody-antigen complex with an antibody that specifically binds the antigen under suitable incubation conditions described above. The amount of an antibody-antigen complex can be determined by comparing the measured value to a standard or control. The AUC for the antigen (biomarker) can then be calculated using techniques known, such as, but not limited to, a ROC analysis.

### Multiplex Tissue Analysis

In standard IHC in which one biomarker is analyzed per tissue section, there may not be sufficient tissue present in serial sections of tissue to analyze 5-10 biomarkers especially in core needle biopsies that have scant numbers of cancer cells. Methods utilizing IHC can provide additional information (e.g. morphology, location of biomarkers) which can be important when analyzing biomarkers in a solid tumor. Such methods included layered immunohistochemistry (L-IHC), layered expression scanning (LES) or multiplex tissue immunoblotting (MTI) taught, for example, in U.S. Pat. Nos. 6,602,661, 6,969,615, 7,214,477 and 7,838,222; U.S. Publ. No. 2011/0306514 (incorporated herein by reference); and in Chung & Hewitt, Meth Mol Biol., Prot Blotting Detect, Kurlen & Scofield, eds. 536:139-148, 2009, each reference teaches making up to 8, up to 9, up to 10, up to 11 or more images of a tissue section on layered and blotted membranes, papers, filters and the like, can be used. Coated membranes useful for conducting the L-IHC/MTI process are available from 20/20 GeneSystems, Inc. (Rockville, Md.).

In lieu of L-IHC, other multiplex tissue analysis techniques might also be useful for identifying optimal biomarkers according to the present invention. Such techniques should permit at least five, or at least ten or more biomarkers to be measured from a single formalin-fixed, paraffin-embedded (FFPE) section due to the frequent scarcity of pre-treatment samples (especially needle biopsies). Furthermore, for reasons stated above, it is advantageous for the technique to preserve the localization of the biomarker and be capable of distinguishing the presence of biomarkers in cancerous and non-cancerous cells.

The L-IHC method can be performed on any of a variety of tissue samples, whether fresh or preserved. For example, in the studies exemplified below, prostate cancer assays were performed on samples from pathology tissue archives received after IRB approval of the protocol. The samples included core needle biopsies that were routinely fixed in 10% normal buffered formalin and processed in the pathology department. Standard five μm thick tissue sections were cut from the tissue blocks onto charged slides that were used for L-IHC. Expression of multiple biomarkers can be correlated with early stage aggressive CaP.

Thus, L-IHC enables testing of multiple markers in a tissue section by obtaining copies of molecules transferred from the tissue section to plural bioaffinity-coated membranes to essentially produce copies of tissue “images.” In the case of a paraffin section, the tissue section is deparaffinized as known in the art, for example, exposing the section to xylene or a xylene substitute such as NEO-CLEAR®, and graded ethanol solutions. The section can be treated with a proteinase, such as, papain, trypsin, proteinase K and the like. Then, a stack of a membrane substrate comprising, for example, plural sheets of a 10 μm thick coated polymer backbone with 0.4 μm diameter pores to channel tissue molecules, such as, proteins, through the stack, then is placed on the tissue section. The movement of fluid and tissue molecules is configured to be essentially perpendicular to the membrane surface. The sandwich of the section, membranes, spacer papers, absorbent papers, weight and so on can be exposed to heat to facilitate movement of molecules from the tissue into the membrane stack. A portion of the proteins of the tissue are captured on each of the bioaffinity-coated membranes of the stack (available from 20/20 GeneSystems, Inc., Rockville, Md.). Thus, each membrane comprises a copy of the tissue and can be probed for a different biomarker using standard immunoblotting techniques, which enables open-ended expansion of a marker profile as performed on a single tissue section. As the amount of protein can be lower on membranes more distal in the stack from the tissue, which can arise, for example, on different amounts of molecules in the tissue sample, different mobility of molecules released from the tissue sample, different binding affinity of the molecules to the membranes, length of transfer and so on, normalization of values, running controls, assessing transferred levels of tissue molecules and the like can be included in the procedure to correct for changes that occur within, between and among membranes and to enable a direct comparison of information within, between and among membranes. Hence, total protein can be determined per membrane using, for example, any means for quantifying protein, such as, biotinylating available molecules, such as, proteins, using a standard reagent and method, and then revealing the bound biotin by exposing the membrane to a labeled avidin or streptavidin; a protein stain, such as, Blot fastStain, Ponceau Red, brilliant blue stains and so on, as known in the art.

In one embodiment, the present methods utilize Multiplex Tissue Imprinting (MTI) technology for measuring biomarkers, wherein the method conserves precious biopsy tissue by allowing multiple biomarkers, in some cases at least six biomarkers, to be tested on a single 5 micron section of biopsy tissue. See Example 1.

In other embodiments, alternative multiplex tissue analysis systems exist that may also be employed as part of the present invention. One such technique is the mass spectrometry-based Selected Reaction Monitoring (SRM) assay system (“Liquid Tissue” available from OncoPlexDx (Rockville, Md.). That technique is described in U.S. Pat. No. 7,473,532.

Another is the multiplex IHC technique developed by GE Global Research (Niskayuna, N.Y.). That technique is described in U.S. Pub. Nos. 2008/0118916 and 2008/0118934. There, sequential analysis is performed on biological samples containing multiple targets including the steps of binding a fluorescent probe to the sample followed by signal detection, then inactivation of the probe followed by binding probe to another target, detection and inactivation, and continuing this process until all targets have been detected.

Another system that might be employed is the AQUA software system available from HistoRx (Branford, Conn.).

In other embodiments, multiplex tissue imaging can be performed when using fluorescence (e.g. fluorophore or Quantum dots) where the signal can be measured with the multispectral imagine system Nuance™ (Cambridge Research & Instrumentation, Woburn Mass.). As another example, fluorescence can be measured with the spectral imaging system SpectrView™ (Applied Spectral Imaging, Vista, Calif.). Multispectral imaging is a technique in which spectroscopic information at each pixel of an image is gathered and the resulting data analyzed with spectral image-processing software. For example, the Nuance system can take a series of images at different wavelengths that are electronically and continuously selectable and then utilized with an analysis program designed for handling such data. The Nuance system is able to obtain quantitative information from multiple dyes simultaneously, even when the spectra of the dyes are highly overlapping or when they are co-localized, or occurring at the same point in the sample, provided that the spectral curves are different. Many biological materials auto fluoresce, or emit lower-energy light when excited by higher-energy light. This signal can result in lower contrast images and data. High-sensitivity cameras without multispectral imaging capability only increase the autofluorescence signal along with the fluorescence signal. Multispectral imaging can unmix, or separate out, autofluorescence from tissue and, thereby, increase the achievable signal-to-noise ratio.

Another system that may be used includes reverse phase protein microarrays (RPMA), which are designed for quantitative, multiplexed analysis of proteins, and their posttranslational modified forms, from a limited amount of sample (Chiechi et al. Biotechniques 2012 September; PCT Publication No. WO 2007/047754).

In such multiplex assays, any of a number of different reporters can be used, such as, fluorescence molecules, chemiluminescence molecules, colloidal particles, such as, those carrying a metal, such as, gold, quantum dots (see, for example, US Publ. No. 2001/0023078, and U.S. Pat. Nos. 6,322,901 and 7,682,789), enzymes, which will require a substrate that on reaction yields a detectable signal, and so on, as a design choice, and as known in the art.

Accordingly, in certain aspects, labels are used on the antigen or probe used to bind the biomarkers. In that way, a fluorescent intensity signal is obtained to quantitate the biomarker. Labels include, but are not limited to: light-emitting, light-scattering, and light-absorbing compounds which generate or quench a detectable fluorescent, chemiluminescent, or bioluminescent signal (see, e.g., Kricka, L., Nonisotopic DNA Probe Techniques, Academic Press, San Diego (1992) and Garman A., Non-Radioactive Labeling, Academic Press (1997).). Fluorescent reporter dyes useful as labels include, but are not limited to, fluoresceins (see, e.g., U.S. Pat. Nos. 5,188,934, 6,008,379, and 6,020,481), rhodamines (see, e.g., U.S. Pat. Nos. 5,366,860, 5,847,162, 5,936,087, 6,051,719, and 6,191,278), benzophenoxazines (see, e.g., U.S. Pat. No. 6,140,500), energy-transfer fluorescent dyes, comprising pairs of donors and acceptors (see, e.g., U.S. Pat. Nos. 5,863,727; 5,800,996; and 5,945,526), and cyanines (see, e.g., WO 9745539), lissamine, phycoerythrin, Cy2, Cy3, Cy3.5, Cy5, Cy5.5, Cy7, FluorX (Amersham), Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, Tetramethylrhodamine, and/or Texas Red, as well as any other fluorescent moiety capable of generating a detectable signal. Examples of fluorescein dyes include, but are not limited to, 6-carboxyfluorescein; 2′,4′,1,4,-tetrachlorofluorescein; and 2′,4′,5′,7′,1,4-hexachlorofluorescein. In certain aspects, the fluorescent label is selected from SYBR-Green, 6-carboxyfluorescein (“FAM”), TET, ROX, VICTM, and JOE. For example, in certain embodiments, labels are different fluorophores capable of emitting light at different, spectrally-resolvable wavelengths (e.g., 4-differently colored fluorophores); certain such labeled probes are known in the art and described above, and in U.S. Pat. No. 6,140,054. A dual labeled fluorescent probe that includes a reporter fluorophore and a quencher fluorophore is used in some embodiments. It will be appreciated that pairs of fluorophores are chosen that have distinct emission spectra so that they can be easily distinguished.

In another embodiment, gene expression of markers (e.g. mRNA) is measured in a sample from a human subject. For example, gene expression profiling methods for use with paraffin-embedded tissue include quantitative reverse transcriptase polymerase chain reaction (qRT-PCR), however, other technology platforms, including mass spectroscopy and DNA microarrays can also be used. These methods include, but are not limited to, PCR, Microarrays, Serial Analysis of Gene Expression (SAGE), and Gene Expression Analysis by Massively Parallel Signature Sequencing (MPSS).

Any methodology that provides for the measurement of a marker or panel of markers from a human subject is contemplated for use with the present methods. In certain embodiments, the sample from the human subject is a tissue section such as from a biopsy. In another embodiment, the sample from the human subject is a bodily fluid such as blood, serum, plasma or a part or fraction thereof. In other embodiments, the sample is a blood or serum and the markers are proteins measured there from. In yet another embodiment, the sample is a tissue section and the markers are proteins or mRNA expressed therein. Many other combinations of sample forms from the human subjects and the form of the markers are contemplated.

### Image Analysis

In the case of IHC or L-IHC, using, for example, fluorescent reporters and dyes, automated detection systems can be used to digitize images, to facilitate the process and which can enable a quantitative metric for analysis and comparison. There are several pathology imaging devices on the market including the Biolmagene iScan Coreo system and the widely-used Aperio Scanscope system that can produce digital images of H&E stained as well as fluorescently-labeled slides. Other scanners include the 3D Histech Pannoramic SCAN system that images fluorescently-labeled slide, and the Dako ACIS system for brightfield imaging of slides. Fluorescently-labeled membranes can be scanned on the Typhoon Trio Plus system and image analysis performed using the Autoquant software. The Olympus VS110 Scanning system using OlyVIA software produces digital images of H&E-stained tissue and is best suited for producing digital fluorescent images from membranes. Image analysis can then be performed using the Visiomorph image analysis software available from Visiopharm (Denmark).

### Analysis of Biomarkers

Once measured, the statistical measurement for each biomarker in a given panel is analyzed either individually (univariately) or in aggregate (multivariately) from a panel to provide a probability or likelihood of cancer. For example one might utilize statistical software tools to apply Logistic Regression of Cox Proportional Hazards Regression [applied to time-dependent criteria for disease outcomes] to achieve this end. Alternatively, such high dimensional data analysis might comprise more complex tools such as Differential Dependency Networks (DDN) or other such computational tools to combine clinicopathological, morphological and molecular biomarkers to make predictions. In certain embodiments, the likelihood of cancer is the probability a patient has an early stage aggressive CaP that was originally categorized as Very Low or Low but should be categorized, for the purposes of recommending management of the disease, as Intermediate or High risk for developing aggressive CaP.

In certain embodiments, the probability or likelihood of early stage aggressive CaP or developing aggressive CaP is represented as a percentage the tested patient is positive for the presence of early stage aggressive CaP—their risk of developing aggressive CaP. The outcome of being re-categorized from Very Low or Low to Intermediate or High risk is clinical intervention in the form of surgery, radiation, chemotherapy or other methods for actively controlling and managing the disease.

In certain embodiments, the probability of recurrence or early stage aggressive CaP or developing aggressive CaP is represented as a probability cutoff, wherein above the cutoff the likelihood is positive and below the likelihood is negative. The cutoff may also be referred to as a threshold value wherein the value may be determined methods known in the art including a training set and risk matched controls and an accepted probability, such as 60% or greater. In that way, the biomarker or panels are trained to distinguish indolent from aggressive CaP wherein a calculated value from a tested sample above the threshold value a sample is considered likely positive for aggressive CaP. Value below the threshold value may be considered likely negative for aggressive CaP.

In certain embodiments the probability of cancer is calculated using standard statistical analysis well known to one of skill in the art wherein the measurements of each CaP biomarker is analyzed individually or combined to provide a probability of cancer. In one aspect logistic regression analysis and/or multivariate logistic regression analysis is used to derive a mathematical function with a set of variables corresponding to each marker, which provides a weighting factor for each biomarker. The weighting factors are derived to optimize the agency of the function to predict the dependent variable, which for example may be the continuous (protein biomarker), dichotomous (protein biomarkers), categorical (Gleason score, clinical stage, pathological stage) etc. variable in the patients. The weighting factors are specific to the particular biomarker or combination (e.g. panel) analyzed. The function can then be applied to original samples to predict a probability. In this way, a retrospective data set is used to provide weighting factors for a particular CaP biomarker or panel of CaP biomarkers, which is then used to calculate the probability of early stage aggressive CaP in a patient where the outcome of aggressive CaP is unknown or categorized as Very Low or Low prior to screening using the present methods.

Other established methods may also be used to analyze the measurement data from the CaP biomarkers in a patient sample to either diagnose aggressive CaP and/or determine the likelihood a patient has early stage aggressive CaP cancer and/or determining risk a patient has for developing aggressive CaP cancer and/or determining the increase (e.g. Intermediate or High) in risk of developing aggressive CaP to a patient.

US Publ. No. 2008/0133141 (herein incorporated by reference) teaches statistical methodology for handling and interpreting data from a multiplex assay. The amount of any one marker generates a βeta coefficient which is combined with a model constant and to produce a solution that can be compared to a predetermined model cutoff (e.g. 80% specificity) for distinguishing positive from negative for that marker as determined from a control population study of patients with cancer and suitably risk matched normal controls to yield a score for each marker based on said comparison; and then combining the scores for each marker to obtain a composite score for the marker(s) in the sample.

A predetermined cutoff can be based on ROC curves and the score for each marker can be calculated based on the specificity of the marker. Then, the total score can be compared to a predetermined total score to transform that total score to a qualitative determination of the likelihood or risk of developing aggressive CaP.

One benefit of the present method is identifying those patients previously categorized as Very Low or Low risk for developing aggressive CaP wherein the recommended management is surveillance, who in fact should be categorized as Intermediate or High risk for developing aggressive CaP, wherein the recommendation is clinical intervention. That may take the form of surgery, radiation, chemotherapy or other methods to control the progression of the disease.

Once the physician or healthcare practitioner understands a patient has been categorized as Intermediate or High risk for developing aggressive CaP they can recommend, in particular, that those at a higher risk be provided with appropriate treatment. It should be appreciated that the precise numerical cut off above which clinical intervention is recommended may vary depending on many factors including, without limitation, (i) the desires of the patients and their overall health and family history, (ii) practice guidelines established by medical boards or recommended by scientific organizations, (iii) the physician's own practice preferences, and (iv) the nature of the biomarker test including its overall accuracy and strength of validation data.

Combining histomorphometry of cellular and tissue structure and biomarkers associated with aggressive CaP provides a novel algorithm to determine if active surveillance CaP patients can remain on AS or if they would benefit from clinical intervention.

### EXAMPLES

### Example 1—Exemplary MTI Protocol

We first optimized all MTI primary antibodies (FIGS. 1 and 2) of all our 6 biomarkers on a test TMA with PCa. Our results show that proteins on a single 5 μm FFPE tissue slide could be successfully transferred onto a series of 6 membranes and then be probed with 6 biomarkers simultaneously (FIG. 2). The intensity of biomarker fluorescence signal correlates well with corresponding protein expression.

The tissue slides were deparaffinized and the biopsy or tissue microarrays (TMA) tissues were subjected to enzyme digestion by a cocktail of trypsin and proteinase K solution and heat for antigen retrieval.

During the transfer, (i) a spacer membrane, (ii) a stack of P-Film membranes, (iii) a nitrocellulose membrane trap (iv) 3MM paper (v) were placed on the slide in this order. In the bottom of the slide an absorbent pad and a glass slide support were also placed.

Before placing all membranes, 3MM paper and absorbent pads were equilibrated in transfer buffer (Note: P-Film membranes have two different sides: glossy and nonglossy sides and the glossy side of the membrane stack should face the tissue on the slide). The transfer assembly unit was placed in a Kapak Seal PAK pouch, heat sealed by an impulse sealer and subjected to heat facilitated protein transfer using a heat block.

Once the transfer was completed, excess transfer buffer was removed by washing the membranes in PBS. The nitrocellulose and the spacer membranes were stained using the Blot FastStain kit to confirm the protein transfer.

Then the membranes were treated with streptavidin chemically linked through amino groups and labeled with Cy5 (for total protein).

Next, FITC conjugated anti-rabbit or anti-mouse or anti-goat IgG (appropriate to the primary antibody) in antibody dilution buffer. After properly washing the membranes, each membrane was by adding of an appropriately diluted primary antibody labeled with FITC in a dilution buffer incubated overnight (16-18 h) at 4° C. in a Kapak SealPAK pouch.

The membranes were dried after the final wash between two sheets of 3MM paper.

### Example 2—Biomarker Targets

The following is a discussion of at least 6 biomarkers (including three forms of ProPSA for a total of 8 biomarkers), that based on published data or the Applicants own work, that either indicate a correlation with aggressive CaP and therefore the ability to correctly identify and distinguish samples in a risk category, or is a proliferation marker which can be used as a control when testing tissue samples.

Periostin (POSTN):

Quantitative glycoproteomics analysis of prostate cancer tissues from indolent and lethal tumors using solid-phase glycopeptide extraction method were first conducted. After labeling glycopeptides with iTRAQ 8-plex, the inventors analyzed the samples with LTQ Orbitrap Velos and identified forty-two glycoproteins associated with lethal CaP.

Periostin (POSTN) was further analyzed in primary prostate tumors using chromagen-based (DAB) IHC. Periostin is a component of the extracellular matrix expressed by fibroblasts in normal tissues and stroma of primary tumors. The metastatic colony formation requires the induction of periostin in the foreign stroma by the infiltrating cancer cells. The staining of POSTN exhibited a low background in the normal prostate samples but revealed an overexpression in the peritumoral stroma of Gleason grade 3 tumors, which seldom result in metastases, and strong overexpression in the peritumoral stroma of Gleason grade 4 tumors, which commonly result in metastases. This indicates that periostin expression may correlate with aggressive CaP.

Calcium Channel, Voltage Dependent, I Type, Alpha 1d Subunit (CACNA1D):

Washington and Weigel (13) found that Vitamin D receptor (VDR) activation induces TMPRSS2 expression in LNCaP prostate cancer cell (metastatic lymph node cell line). They showed that the natural VDR agonist 1α25-dihydroxyvitamin D3 and its synthetic analog EB1089 increase expression of TMPRSS2:ERG mRNA in VCaP cells (metastatic vertebral cell line) resulting in increased ETS-related gene (ERG) protein expression and ERG activity as demonstrated by an increase in the ERG target gene CACNAM.

The inventors have unpublished data that CACNAM (a glycoprotein) is over-expressed at the mRNA level by qRT-PCR. Further, the CACNA1D protein by Western blot is over-expressed in the CaP cell lines (LNCaP, DU145, and PC3) but not in benign prostatic hyperplasia (BPH) and normal epithelium (PrEC) cell lines. In addition use of detection antibody in IHC was positive for overexpression of CACNA1D protein in biopsy samples. This indicates that CACNAM protein expression may correlate with aggressive CaP.

Her-2/Neu Oncogene Over-Expression and DNA Content:

The inventors previously demonstrated that Her-2/neu as well as DNA content was increased significantly in biochemical progression, metastasis and CaP-specific survival. The Fisher Biomarker & Biorepository Laboratory at the Johns Hopkins Medical Institutions (JHMI FBBL) previously showed in a cohort of 124 patients with mean follow-up duration of 6.6 years reported that Her-2/neu expression is a predictor of CaP progression. We confirmed our observations of prognostic value of Her-2/neu expression and DNA content in 252 clinically localized CaP patients with long-term follow-up after radical prostatectomy for progression, metastasis and CaP-specific death.

EZH2:

EZH2 is designated as an epigenetic regulated marker of progressive disease in many solid tumors, including prostate and breast cancer. Laitinen S et al. in 2008 demonstrated that EZH2, Ki-67 and MCM7 are prognostic biomarkers in prostatectomy treated patients [53]. The author's assessed 86 CaP cases of low grade (Gleason score less than 7.0) for the ability of MCM7, Ki67 and EZH2 to predict PSA recurrence, and only Ki67 was significant (p=0.005).

PSA derivatives (i.e. freePSA and (−2,−5,−7)ProPSA) in serum and tissue have predictive capacity as it relates to CaP outcomes. ProPSA is the precursor form of PSA and contains a 7 amino acid pro leader peptide. Additional truncated forms of ProPSA also exist in serum, primarily those with leader sequences of 5, 4 and 2 amino acids. Cleavage activity of the leader sequences by human kallikrein 2 and trypsin activates the PSA protein. The JHMI FBBL group showed that [−5−7] ProPSA evaluated in cancer and benign adjacent tissue areas (BAA) by quantitative immunohistochemistry (IHC), the stain intensity, [p=0.003] were significant predictors of an unfavorable AS biopsy conversion in both Kaplan-Meier and Cox regression analysis.

Ki67 is a proliferative biomarker for cancer. The marker, however, did not demonstrate a correlation with CaP outcomes, but as a proliferation marker Ki67 may find use as a control.

### Example 3—Summary of the Six Biomarker Results Obtained Using the MTI Protocol (TMA 681 and 682) for Both Cancer and Non-Cancer Areas

Two tissue microarrays (TMAs; 681 and 682) including 22 cases with BCR with a mean of ten years follow-up among the total 80 cases were constructed. The BCR cases compromise an increase in PSA, local recurrence of prostate cancer, distant metastasis, both local recurrence and distant metastasis, decrease in PSA through radiation treatment. The detailed information of BCR among the 80 cases are shown in FIG. 35. The detailed demographics of the total 80 cases separated by recurrences are shown in FIG. 36 and further sub-grouped based on recurrence. In the 80 cases, one patient in TMA 682 died from non-cancer cause; there is no follow-up data in 5 cases of TMA 681 and 5 cases of TMA 682.

TMA 681 and 682 (N=80 CaP cases with Gleason score of 6 (n=10); 7 (n=40); 8 (n=20) and 9 (n=10)) were used to test the six different biomarkers from Example 2, and each CaP case includes quadruplicates of cancer and cancer-adjacent benign areas. GraphPad Prism 6 software (La Jolla, Calif.) was applied for data plots of biomarkers expression in the four test groups with different Gleason scores. one-way ANOVA was utilized for analysis followed by Dunnet's multiple comparisons test to evaluate the four Gleason score groups. For statistical modeling, STATA 13.0 (STATA™, StataCorp LP, College Station, Tex.) was used. Statistical significance was set at p<0.05. Our methodology included logistic regression or backwards stepwise multivariate logistic regression (MLR) to discriminate indolent from aggressive PCa.

Specifically, each transfer membrane was scanned at appropriate wavelength with Scan Array Express Microarray Scanner [excitation at 633 nm induces the Cy5 fluorescence (red emission) for total protein, while excitation at 488 nm induces FITC fluorescence (blue emission)].

The scanned images were analyzed using ImageQuant 5.2 software and the results normalized for the specific Cy5 total protein concentrations (expressed as relative signal of each specific biomarker based on total protein) were analyzed by appropriate statistical tools.

The expression level of each biomarker was normalized to the total protein amounts detected on the same membrane. Biomarker Information collected includes Volume, Area, and Pixel intensity. Results are interpreted as Volume=Pixel Intensity×Area.

The results of the MTI protocol are shown in FIG. 2 for the TMA 681 and 682. The top row of slides shows each membrane of the stack in the MTI procedure after being analyzed with the specific antibody for each biomarker. The bottom row of slides provides a comparison against total protein utilizing the Cy-5 antibody.

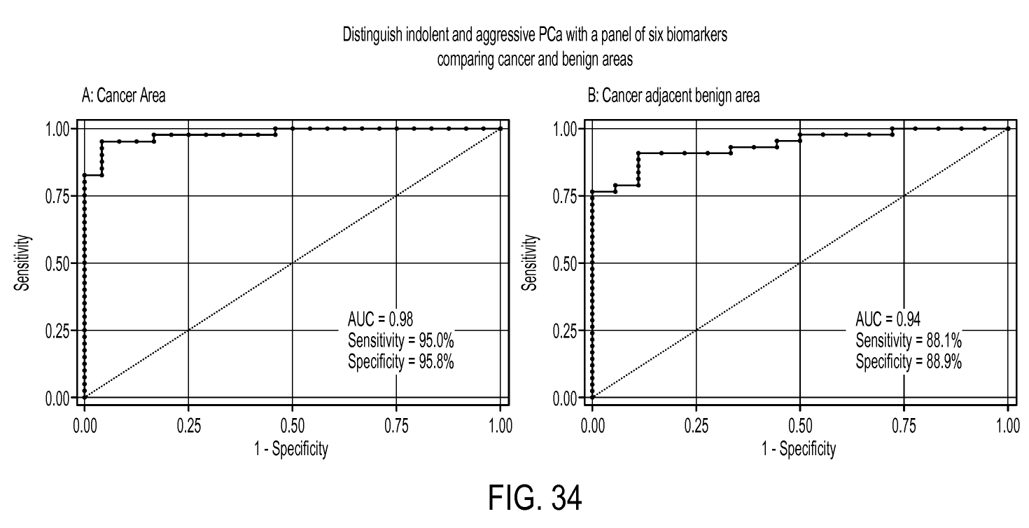

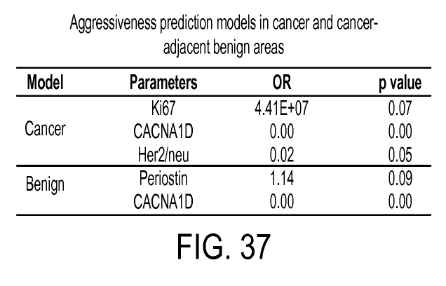

Each biomarker was analyzed individually in both cancer areas (FIGS. 3 to 9) and cancer adjacent benign areas (FIGS. 19 to 25) for the ability to distinguish indolent (Low and Very Low) from aggressive CaP. FIG. 37 shows the model for each marker (cancer area or cancer adjacent benign areas) and their respective odds rations and p values. The markers were further analyzed as a panel of six biomarkers in cancer and cancer adjacent benign areas (FIG. 34).

In FIG. 34, the indolent PCa, with a primary Gleason score of 3 and secondary Gleason score 3 or 4, (e.g. Low and Very Low Risk category according to Table 1) were compared with aggressive PCa (with primary Gleason score of 4 and secondary Gleason score >=3) (e.g. Intermediate and High Risk category) were analyzed based on measurement of the six biomarkers. Logistic regression was used to distinguish indolent and aggressive PCa using the biomarkers. FIGS. 34 (A&B) PCa cancer area, probability cutoff: 59.4% with a AUC of 0.98 (sensitivity 95% and specificity 95.8%); FIGS. 34(C&D) PCa cancer-adjacent benign area, probability cutoff: 70.9% with an AUC value of 0.94 (sensitivity 90.48% and specificity 88.89%).

Although the intermediate risk group of clinically localized prostate has a Gleason score of 7, it includes Gleason 3+4 and Gleason 4+3, patients with the two pathological statuses have significantly different treatment since most Gleason 3+4 could be managed conservatively while all Gleason 4+3 necessitates immediate intervention with surgery and/or adjuvant therapy. Therefore, the Gleason score 3+3 & 3+4 cases were grouped as less aggressive and Gleason score 4+3 & ≧8 cases as aggressive phenotype. Using multivariate logistic regression (MLR), the results of FIG. 34A show that a model retaining CACNA1D, HER2/neu and Ki67 expression distinguishes less aggressive and aggressive PCa in cancer areas at the cutoff selection of Pr at 0.1. The predictive probability is close to 1.0 for most cases in the aggressive groups (Gleason 4+3 & ≧8) (FIG. 34B). The odds ratios (ORs) of Ki67, CACNA1D, Her2/neu are 4.41e+07, <0.01, and 0.02 respectively (FIG. 37). Interestingly, a MLR model that retained CACNA1D and Periostin expression could also distinguish indolent and aggressive prostate cancer in cancer adjacent benign areas (FIG. 3C). The predictive probability is close to 1.0 for most cases in the aggressive groups (FIG. 3D). The ORs are 1.14 and <0.01, respectively (FIG. 37).

Individual Biomarker Analysis

Contrary to published data, measurement of Periostin on the tissue microarrays 681 and 682, a stromal expressed glycoprotein biomarker, decreases with increasing Gleason scores (FIGS. 3 and 37) for non-transformed and transformed data. Directly below the graphs of FIG. 3 is a table with the AUC values as determined by a ROC analysis, wherein the higher the Gleason score, presented as a Gleason Grade Pattern, the higher the AUC value. The two tables summarize the statistical analysis for both data formats.

The AUC values demonstrate the utility of measuring Periostin to distinguish indolent CaP (those tumors with a Gleason score of 6 or less) from aggressive CaP (those tumors with a Gleason score of 8 or above). In other words, the measurement of Periostin can be used to distinguish between Very Low or Low risk and High risk.

The AUC values also demonstrate the utility of measuring Periostin to identify an Intermediate risk category sample, but with a lower sensitivity than distinguishing between Low and High risk, depending on the Gleason Grade Pattern. For those tumors graded as 4+3, the tumors can be distinguished from a tumor graded as 3+3, but not for a tumor graded as 3+4.

The antigen retrieval methods for MTI and chromagen-based IHC differ considerably and Applicants hypothesize that difference in antigen retrieval is at least partly responsible for the difference in the results observed with the MTI protocol (inverse relationship between Periostin and CaP) as compared to a direct correlation observed between increasing amounts of Periostin and an increasing Gleason score using chromogenic-based IHC. Chromagen-based methods use steam heat and no enzyme fort antigen retrieval. For MTI, the TMA or biopsy tissue slides were deparaffinized and the biopsy or TMA tissues were subjected to enzyme digestion by a cocktail of trypsin and proteinase K solution and dry heat for antigen retrieval. This difference might explain disparate results between chromagen-based (DAB) and MTI methods; the results in Example 2 were obtained with Chromagen-based DAB IHC while those in Example 3 were obtained with the MTI protocol.

The results for Periostin (POSTN) are somewhat different between the two methodologies in terms of the correlation with Gleason grade or score. The data from the TMA 681 and 682 experiments using the MTI protocol shows a decrease of Periostin with an increase in the Gleason score based on our formula of volume=(relative) Intensity×area, where Intensity is adjusted for background and total protein. When the experiment was repeated using only FITC labeled POSTN antibody, the results were the same as with the normalized analysis.

The experiments were repeated twice for both glycoproteins (POSTN and CACNA1D). However, when the measured biomarker was assessed for correlation to the Gleason score there was a good correlation by Pearson's test indicating POSTN and CACNA1D glycoproteins are predictive of biochemical recurrence. Further, when POSTN was measured in Active Surveillance biopsies the results were the same as it relates to selecting Escapees vs. Controls. An “Escapee” is a patient that was assigned to AS but later developed aggressive CaP, a “non-Escapee” is a patient that entered AS and did not develop aggressive CaP. Again, tissue processing methods may help explain these differences.

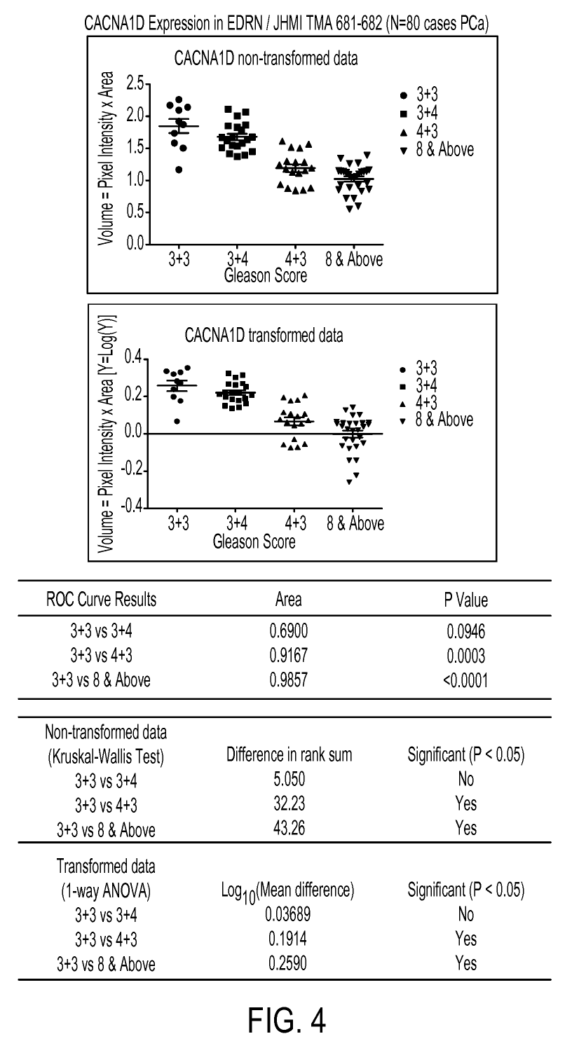

Measurement of CACNA1D, an epithelial expressed glycoprotein biomarker, also decreases with increasing Gleason score as illustrated in (FIG. 4) for non-transformed and transformed data. The AUC values as determined by ROC analysis show a continuous increase in value as the Gleason score, represented as a Gleason Grade Pattern, increases. The two tables summarize the statistical analysis for both data formats and showed 3+3 & 3+4 compared to 4+3 & 8 and demonstrated clear separation for both data formats.

The AUC values demonstrate the utility of measuring CACNAID to distinguish indolent CaP (those tumors with a Gleason score of 6 or less) from aggressive CaP (those tumors with a Gleason score of 8 or above). In other words, the measurement of CACNAID can be used to distinguish between Very Low or Low risk and High risk to re-categorize those patients in the Very Low and Low group that should be in the High risk group.

The AUC values also demonstrate the utility of measuring CACNAID to identify an Intermediate risk category sample, depending on the Gleason Grade Pattern. For those tumors graded as 4+3, the tumors can be distinguished from a tumor graded as 3+3 with a high level of accuracy, but to a lesser extent for a tumor graded as 3+4.

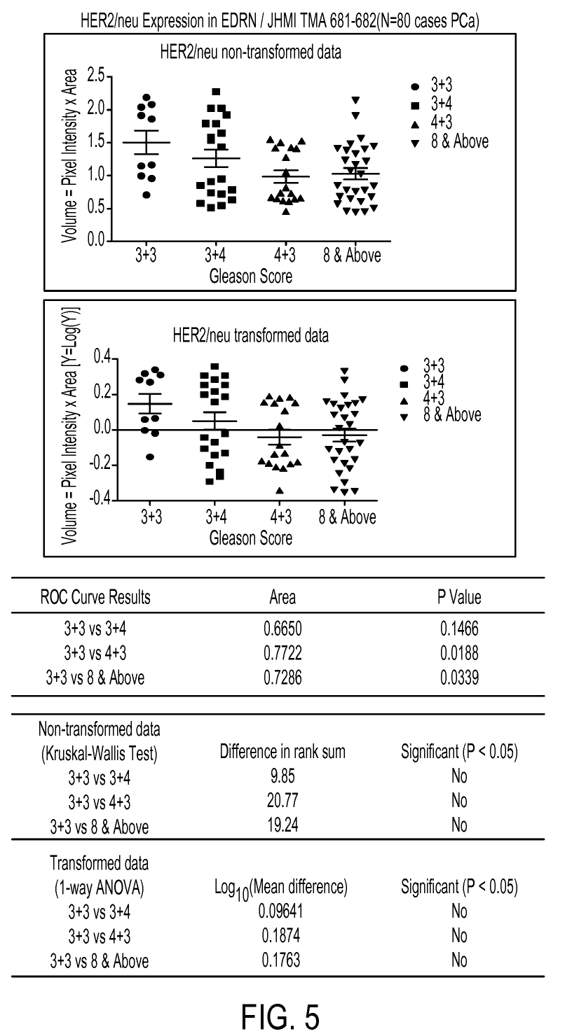

Her2/neu, an epithelial expressed oncogene, was measured and analyzed to determine the utility of distinguishing between Low, Intermediate and High risk categories. AUC values were determined by ROC analysis, wherein an AUC value of 0.73 was calculated for distinguishing samples between Low and High risk and an AUC value of 0.77 was calculated for distinguishing samples between Low and intermediate risk provided the tumor was graded as 4+3. For tumors graded as 3+4 the AUC value distinguishing between Low risk was only 0.67. However the statistical analyses of AUC values of 0.77 and 0.73, were not significant (FIG. 5).

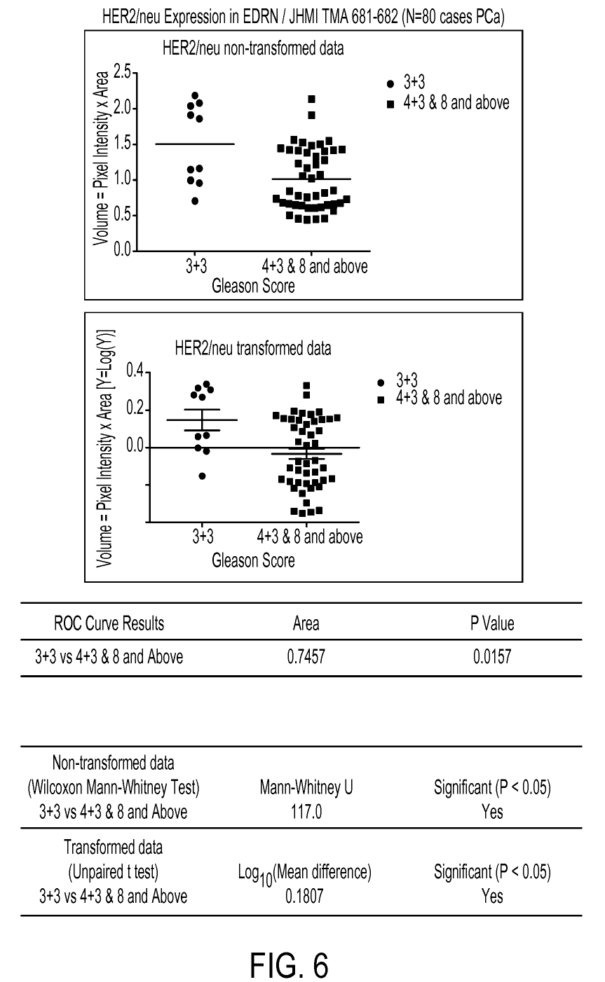

However, when the Intermediate and High risk samples were combined Gleason score (GS) of 3+3 vs. 4+3 and GS 8 and above (FIG. 6) the data was statistically significant with an AUC value of 0.75 using both transformed and non-transformed data formats. Clearly Her2/neu demonstrates utility for distinguishing between indolent and aggressive CaP, however the current data indicates, measuring Her2/neu has less utility for separately identifying Intermediate and High risk samples.

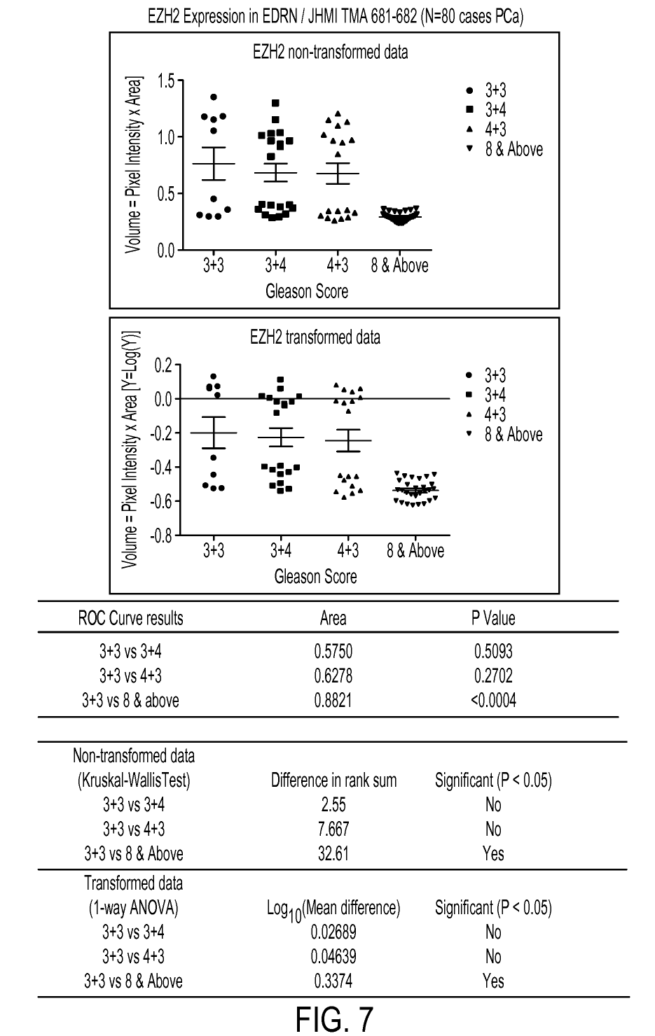

Measuring EZH2, an epithelial expressed epigenetic regulated biomarker, demonstrated utility in distinguishing Low risk samples from High risk with an AUC value of 0.88. (FIG. 7). The AUC values calculated based on a comparison of Low risk (3+3) and Intermediate risk (3+4 or 4+3) samples were not statistically significant. EZH2 represents an aggressive CaP biomarker that demonstrates an ability to distinguish indolent tumors from aggressive CaP tumors.

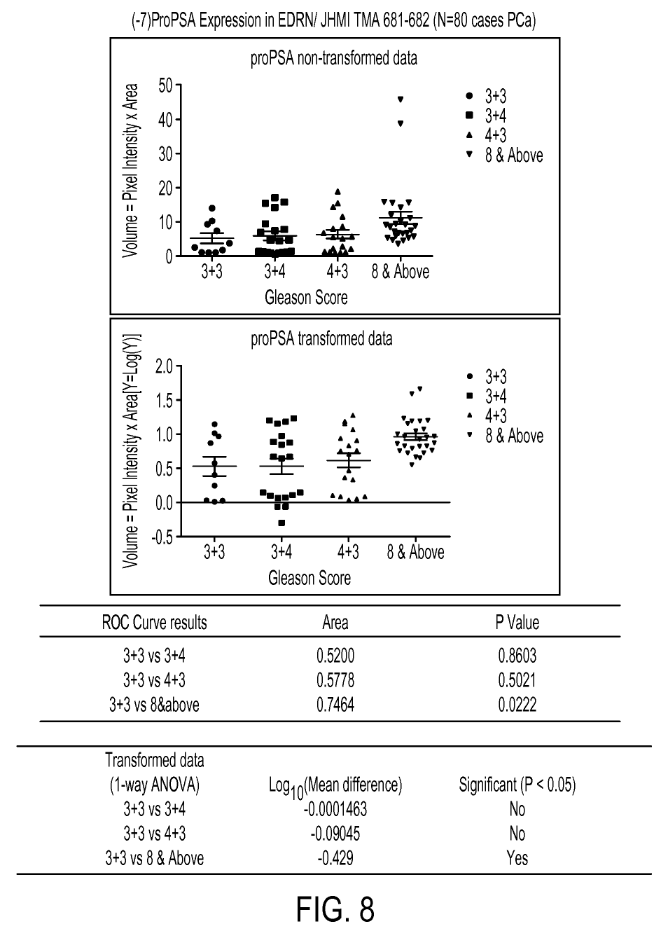

Measuring (−5,−7)ProPSA, an epithelial biomarker and the protein is a truncated form of ProPSA molecule demonstrated utility in distinguishing Low risk samples from High risk samples with an AUC value of 0.75. (FIG. 8). The AUC values calculated based on a comparison of Low risk (3+3) and Intermediate risk (3+4 or 4+3) samples were not statistically significant. This unique PSA derivative (−5,−7)ProPSA represents an aggressive CaP biomarker that demonstrates an ability to distinguish indolent tumors from aggressive CaP tumors.

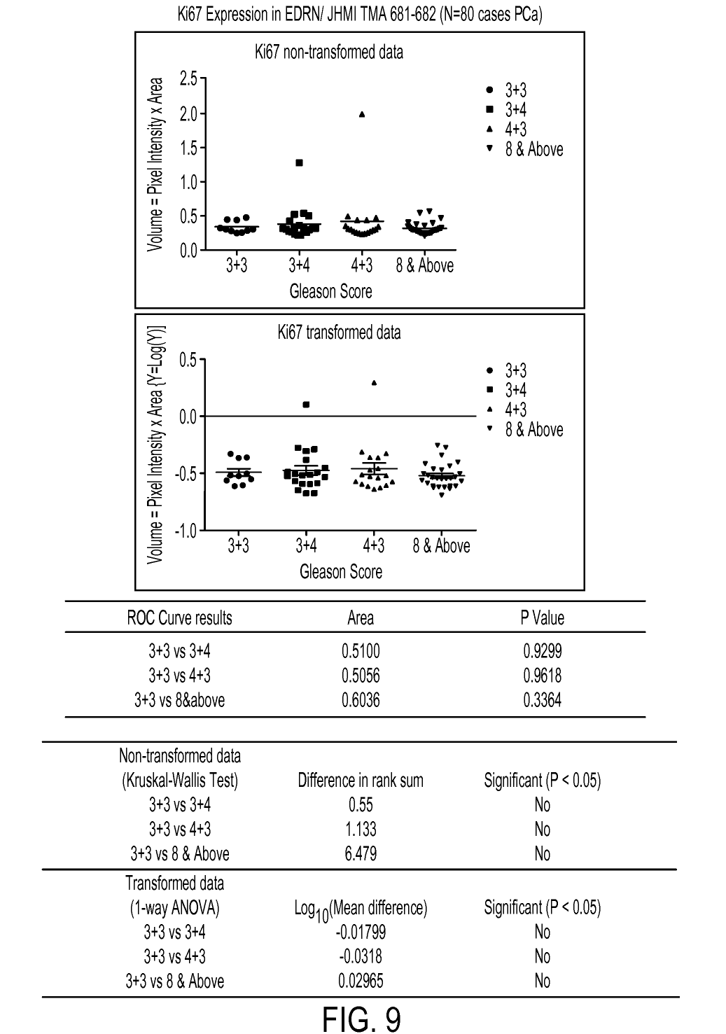

Ki67, an epithelial cell proliferation biomarker, a proliferative biomarker for cancer, showed no significant differences between the different risk group samples (Low, Intermediate and High based on Gleason Scores). See FIG. 9.

The data collected was utilized to identify correlations between each marker and aggressive CaP. In the above-referenced experiments, the correlation analysis for the six biomarkers tested by MTI in TMA 681 & 682 and their Pearson correlation coefficients are provided by the table below:

| | Gleason | | | | | | | PSA |
|---|---|---|---|---|---|---|---|---|
| | Score | CACNA1D | Periostin | EZH2 | Her2Neu | ProPSA | Ki67 | Recurrence |
| Gleason | 1.0000 | | | | | | | |
| score | | | | | | | | |
| CACNA1D | −0.7420 | 1.0000 | | | | | | |
| Periostin | −0.5185 | 0.5590 | 1.0000 | | | | | |
| EZH2 | −0.5253 | 0.5871 | 0.6534 | 1.0000 | | | | |
| Her2Neu | −0.3527 | 0.5419 | 0.6447 | 0.7048 | 1.0000 | | | |
| (−5, −7)ProPSA | 0.3684 | −0.1451 | −0.3672 | −0.4965 | −0.3665 | 1.0000 | | |
| Ki67 | −0.1421 | 0.1905 | 0.2944 | 0.3178 | 0.3967 | −0.1537 | 1.0000 | |
| PSA | 0.3638 | −0.0305 | −0.0522 | −0.0436 | 0.2068 | 0.0632 | −0.0067 | 1.0000 |
| Recurrence | | | | | | | | |

Approximately correlational strength can be interpreted as: 0<|r|<0.3 weak correlation, 0.3<|r|<0.7 moderate correlation, and |r|>0.7 strong correlations.

FIGS. 10 and 27 (cancer area) show the logistic regression to assess biochemical recurrence (BCR) as a binary outcome using only Gleason grade as a categorical variable to predict BCR.

| Recur- | Odds | | | | | |
|---|---|---|---|---|---|---|
| rence | Ratio | Std. Err. | z | P > z | [95% Conf. Interval] | |
| GG | 2.078763 | .5512988 | 2.76 | 0.006 | 1.236124 | 3.495812 |

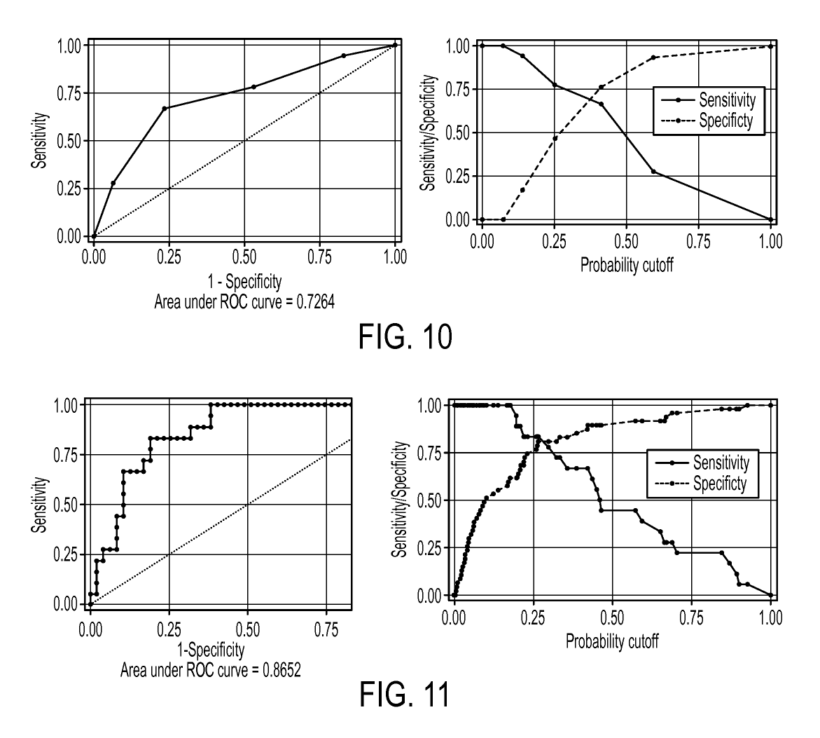

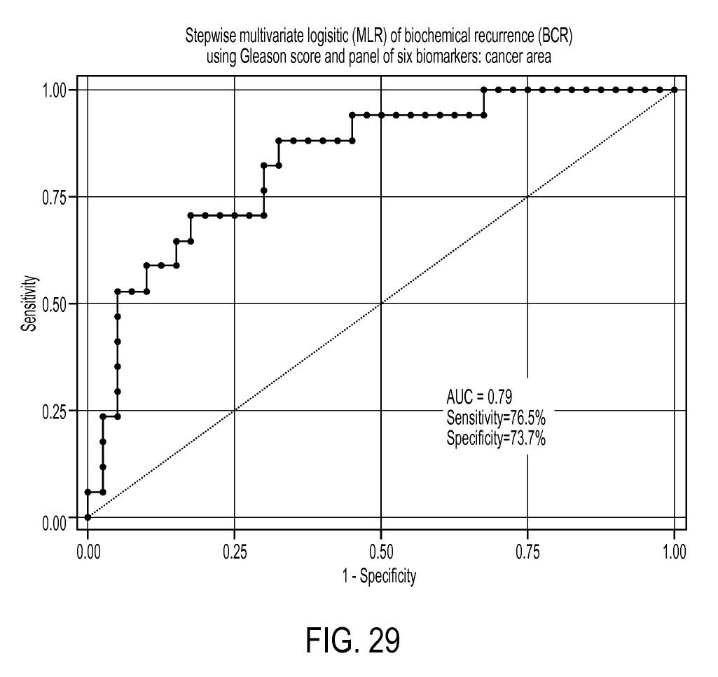

FIGS. 11 and 29 (cancer area) show a Stepwise multivariate logistic regression (MLR) of biochemical recurrence (BCR) using Gleason grade+biomarkers as continuous variables to evaluate BCR: Pr=0.1.

| | Odds | | | | | |
|---|---|---|---|---|---|---|
| Recurrence | Ratio | Std. Err. | z | P > z | [95% Conf. Interval] | |
| GG | 6.567938 | 3.586299 | 3.45 | 0.001 | 2.252405 | 19.15189 |
| CACNA1D | 20.04162 | 31.90202 | 1.88 | 0.060 | .8851166 | 453.8008 |
| Her2Neu | 5.495009 | 4.82802 | 1.94 | 0.052 | .9819434 | 30.75037 |

FIG. 38 is a combination of the data from FIGS. 27 and 29. In the 80 prostate cancer cases, there is no significant difference in age between among the 2 groups of recurrence, see Example 2. Logistic regression using Gleason score alone was able to predict BCR with a ROC-AUC of 0.71 at Pr level of 0.1 (sensitivity=65.00%, specificity=75.51%; cutoff=0.4); while a model that combines Gleason score with CACNA1D and HER2/neu yields a better ROC-AUC (0.79) for the prediction of BCR (sensitivity=52.94% and specificity=76.32%; cutoff=0.4; pr=0.1) (FIG. 38), but there is no significant difference in these two models of BCR prediction (p=0.15). The Combination model could be used to predict recurrence with an Odds Ratio of 6.34, 3.83, 6.77 and 95% CI of 1.90˜21.15, 0.86˜16.99; 0.77˜59.62 for Gleason score, CACNA1D and HER2/neu, respectively).

FIG. 12 shows a stepwise logistic regression of Gleason grade up to 7 vs. above 7 using biomarkers as continuous variable to evaluate Gleason grade: Pr=0.1.

| | Odds | | | | | |
|---|---|---|---|---|---|---|
| GG7 | Ratio | Std. Err. | z | P > z | [95% Conf. Interval] | |
| ProPSA | 1.359936 | .1591449 | 2.63 | 0.009 | 1.081204 | 1.710525 |
| Her2Neu | 69.6352 | 118.182 | 2.50 | 0.012 | 2.501525 | 1938.442 |
| CACNA1D | .0006948 | .0016023 | −3.15 | 0.002 | 7.57e−06 | .0638004 |
| Periostin | .348048 | .2200725 | −1.67 | 0.095 | .1007906 | 1.201872 |

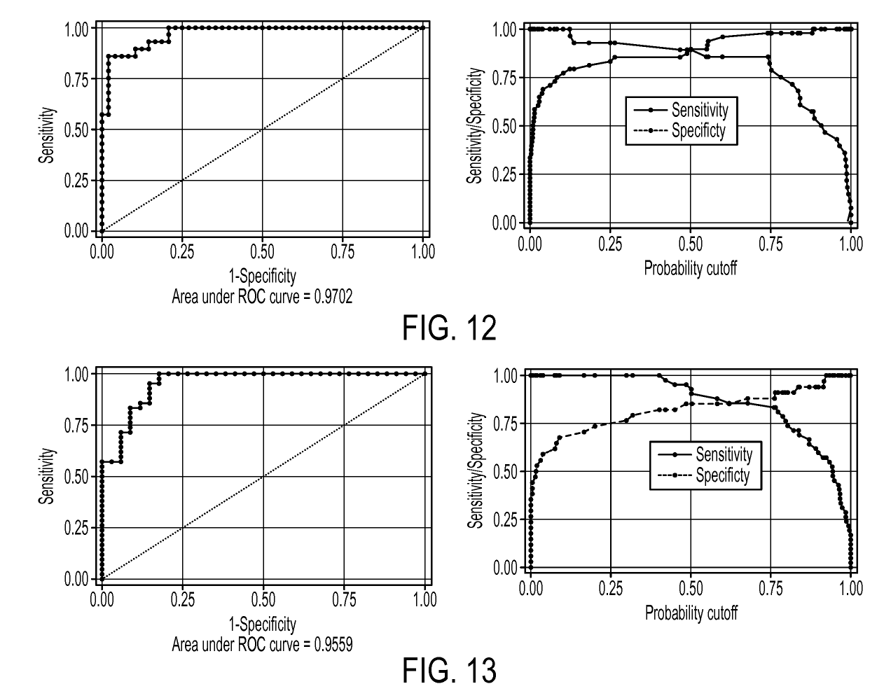

FIG. 13 shows the stepwise logistic regression of Gleason score 3 and 4 (binary) using biomarkers as continuous variables to evaluate Gleason score outcome: Pr=0.1.

| | Odds | | | | | |
|---|---|---|---|---|---|---|
| G3 | Ratio | Std. Err. | z | P > z | [95% Conf. Interval] | |
| Periostin | .102288 | .1022491 | −2.28 | 0.023 | .0144194 | .725609 |
| EZH2 | 5.71e+07 | 4.94e+08 | 2.06 | 0.039 | 2.448783 | 1.33e+15 |
| CACNA1D | .0095027 | .0170209 | −2.60 | 0.009 | .0002839 | .3180436 |

### Example 4: Prediction of Escapee Status with Molecular Markers

Using the biomarkers pre-tested in the TMAs we evaluated a total of 61 AS cases (32 non-escapees and 29 escapees). Biopsies of all the “Escapees” were identified along with a set of controls with long term follow-up from the active surveillance database (over 1500+ total AS cases). This provided the basis for randomly selecting both cohorts (escapees (n=100+) and controls (several hundred), then a pathologist selects biopsies with cancer and sectioned at 0.5 microns for testing. Twenty-one AS biopsies were tested in this process with MTI assay for all six biomarkers individually listed above (FIGS. 14-18) and 29 escapees with 32 non-escapee controls were tested for FIG. 39 (combination of ProPSA and CACNA1D). The first biopsy data to separate “Escapees” from controls is provided below.

FIG. 14A-B:

Periostin compared in Escapees and Controls for 21 cases. Note that two cases had no cancer present in these biopsy samples. An AUC value of 0.66 was calculated comparing Escapees and non-escapees, however it was not statically significant.

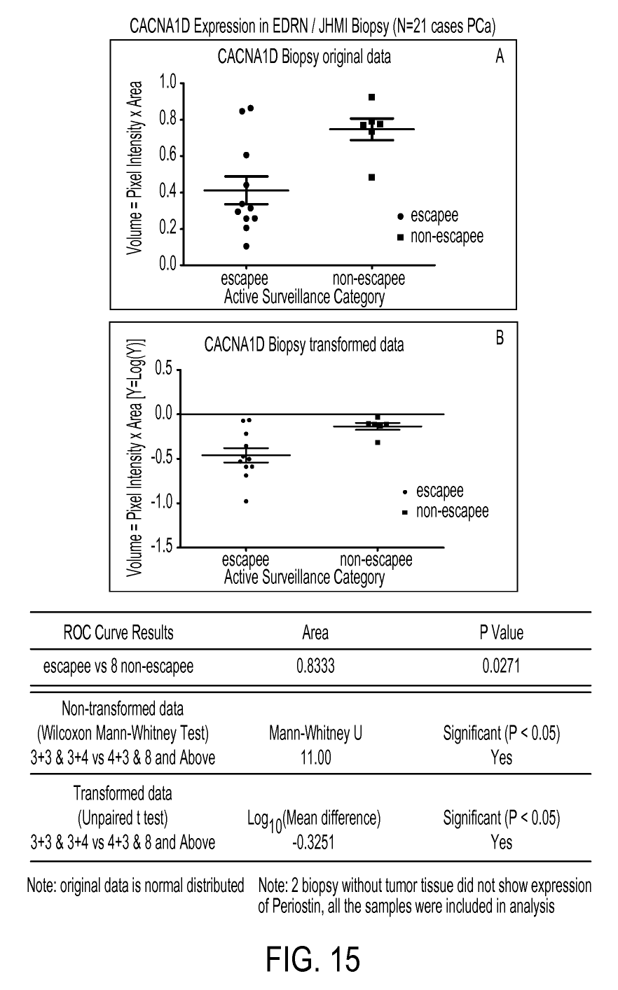

FIG. 15 A-B:

CACNA1D compared in Escapees and Controls for 21 cases. Note that two cases had no cancer present in these biopsy samples. An AUC value of 0.833 was calculated comparing Escapees and non-escapees, demonstrating an excellent biomarker for identifying early stage aggressive CaP that was incorrectly categorized as Very Low or Low risk managed by active surveillance.

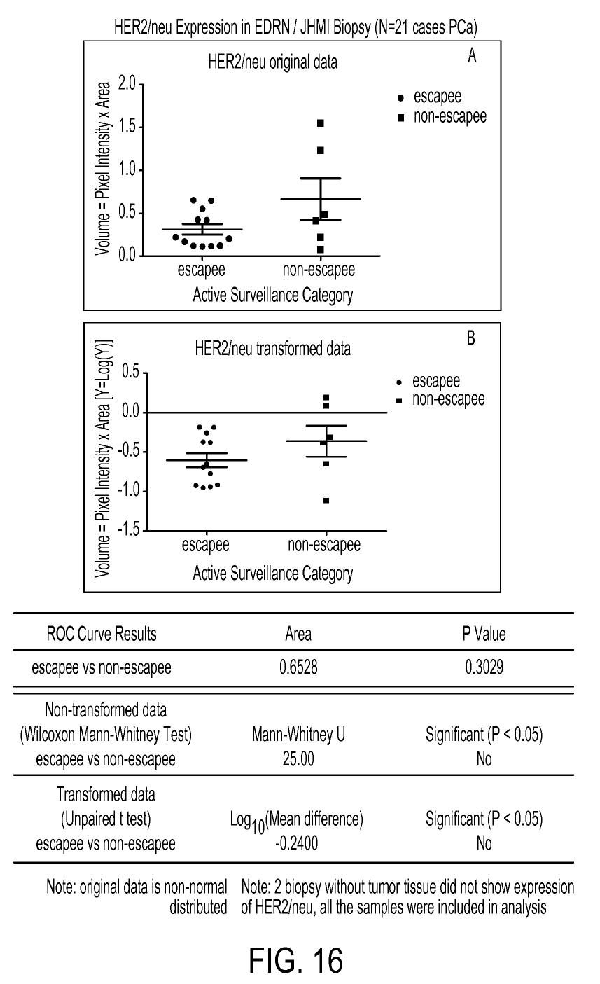

FIG. 16A-B:

Her/2neu compared in Escapees and Controls for 21 cases. Note that two cases had no cancer present in these biopsy samples. An AUC value of 0.65 was calculated comparing Escapees and non-escapees, however it was not statically significant.

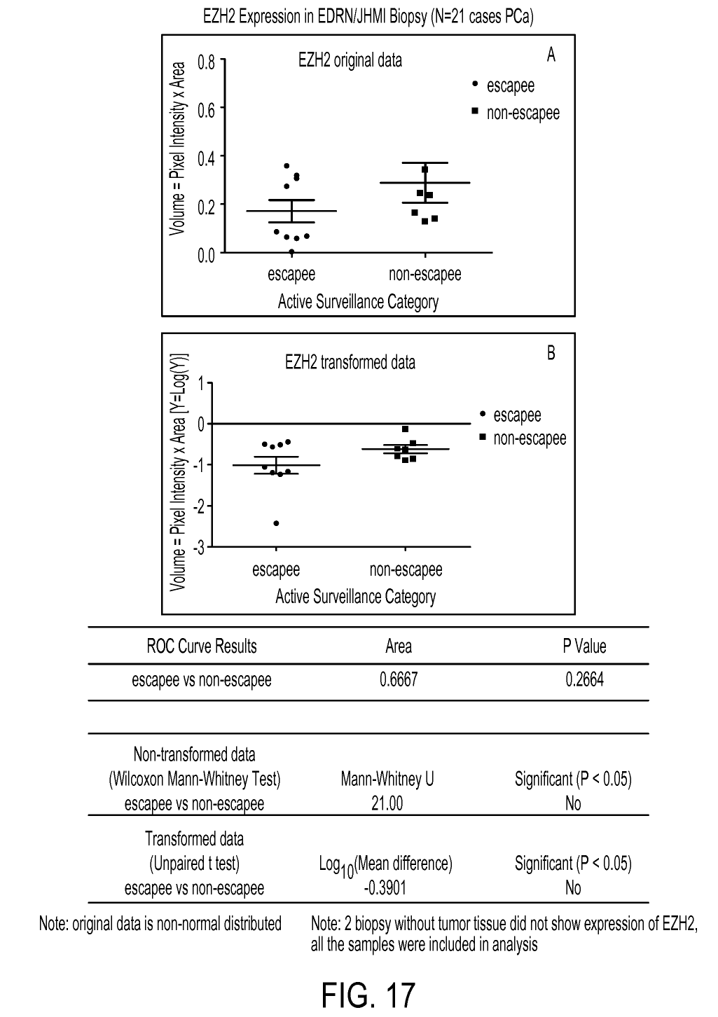

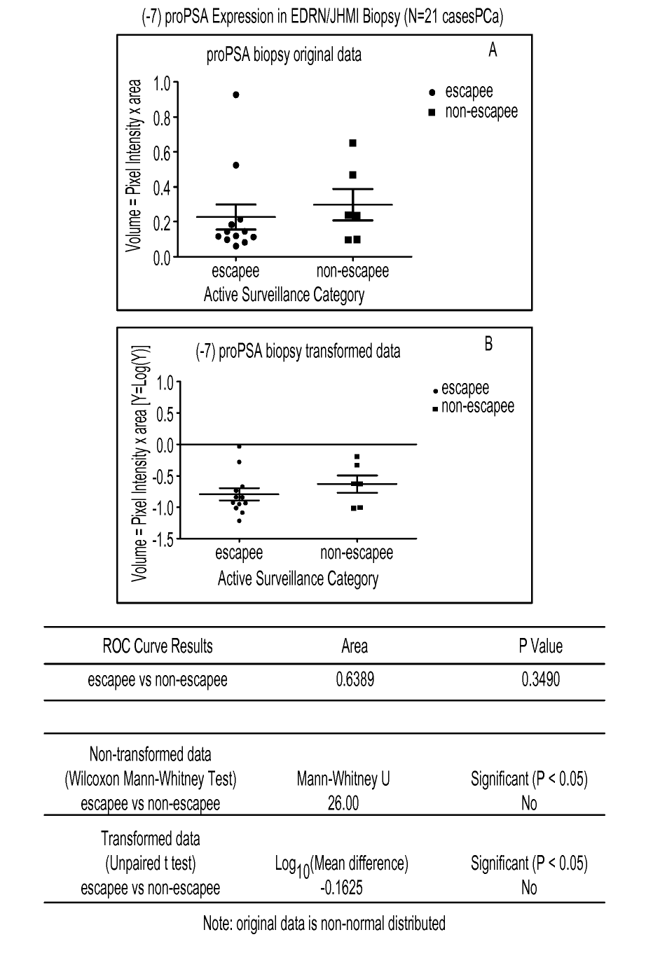

FIG. 17A-B:

EZH2 compared in Escapees and Controls for 21 cases. Note that two cases had no cancer present in these biopsy samples. An AUC value of 0.67 was calculated comparing Escapees and non-escapees, however it was not statically significant.

FIG. 18A-B:

(−7)ProPSA compared in Escapees and Controls for 21 cases. A positive trend pattern is indicated but sample size remains small. An AUC value of 0.64 was calculated comparing Escapees and non-escapees, however it was not statically significant.

FIG. 39:

ProPSA and CACNA1D compared in 29 Escapees and 32 controls. The figure demonstrates the measured biomarkers successfully separated escapees and non-escapees in the AS cohort with ProPSA and CACNA1D, which are retained in a MLR model for molecular biomarkers to predict the AS cases that require definitive treatment. The results illustrate the AUC-ROC of 0.78 and sensitivity (51.72%) and specificity (90.63%) at a Pr level of 0.05 and cutoff of 0.65 (FIG. 5A). The model has an accuracy of 72.13% in the prediction of escapees. The predictive probability of the model was shown in FIG. 39B. The Odds Ratio for (−5,−7)proPSA and CACNA1D are 0.20 and 7.93. The data suggest that CACNA1D alone, or as a subset in combination with ProPSA, have the potential to differentiate escapees from non-escapees patients that can remain in annual monitoring according to the data of 61 biopsy cases with high specificity.

### Example 5

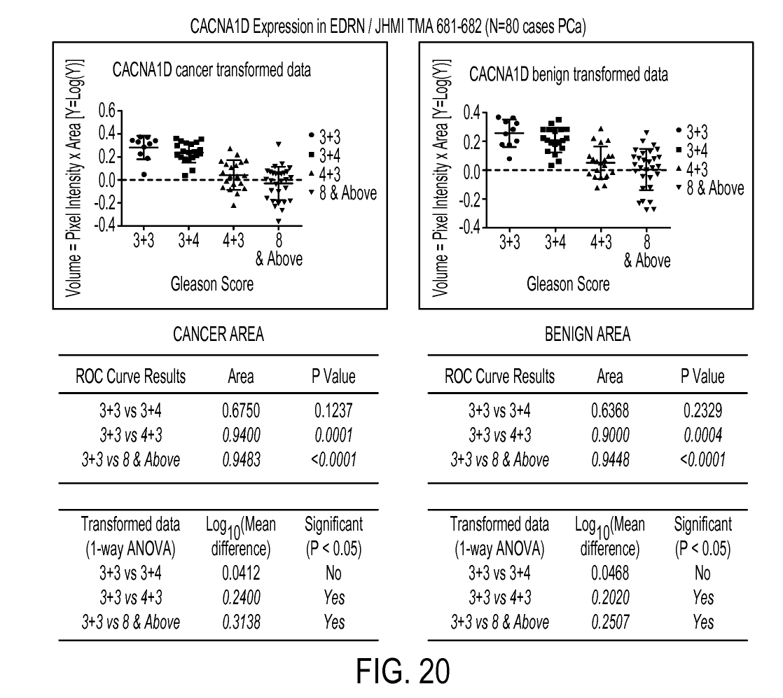

As explained above, the method described herein can be used to identify patients for further treatment based on the difference between the concentration of biomarker in the cancerous and non-cancerous portions of the tissue sample. FIG. 19 shows the graphical representations of Periostin, which shows a significant p value in the benign graph of the results. FIGS. 20 through 25 show similar graphs for each of the other biomarkers. Of the six biomarkers measured, three showed a statistically significant ability to distinguish a 3+3 graded tumors from an 8+ graded tumors. An AUC value of 0.91 was calculated for Periostin expression, as measured in a benign area; an AUC value of 0.94 was calculated for CACNA1D expression, as measured in either a cancer area or benign area of the sample; and an AUC value of 0.79 was calculated for Her2/neu as measured in a cancer area of the sample. CACNA1D was further able to distinguish between tumors graded 3+3 and 4+3 with a calculated AUC value of 0.95 for both cancer and benign area of the sample.

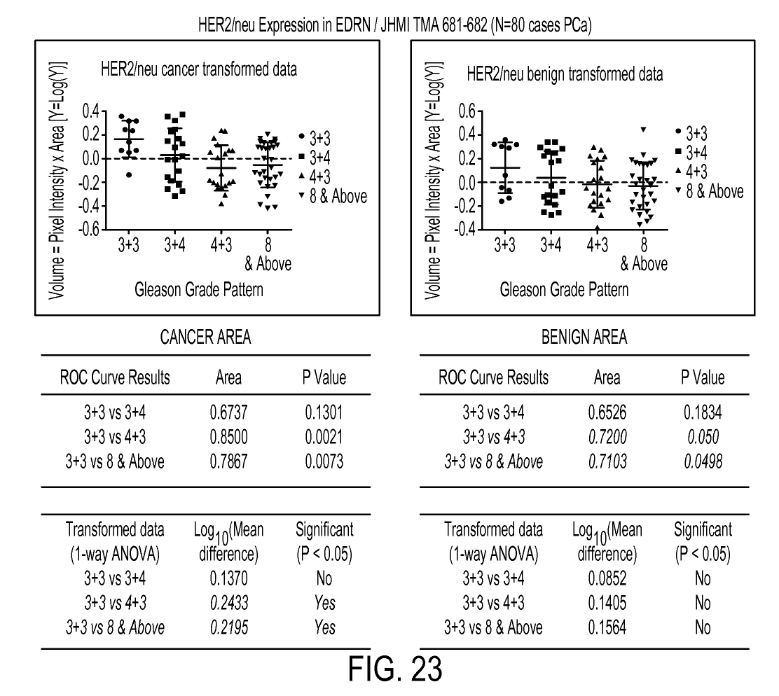

Among the 6 biomarkers, CACNA1D, Her2/neu and Periostin were differentially regulated among these four groups in cancer area and cancer adjacent benign areas. The results show that in cancer areas, CACNA1D and HER2/neu relative expression are significantly lower in groups of Gleason score 4+3, ≧8 compared with Gleason score 3+3 (p<0.05). There is a trend of gradually decreased relative expression of both markers with increased Gleason scores (FIG. 2A-2B). Pearson correlation analysis show that CACNA1D has a strong negative correlation with Gleason score (−0.75) while Her2/neu shows a medium negative correlation (−0.39) with Gleason score. Interestingly, CACNA1D and HER2/neu expression behaves similarly in cancer adjacent benign areas (FIGS. 20 and 23 cancer area vs FIGS. 20 and 23 benign area), suggesting a field effect exists for these markers. Periostin expression was significantly higher in prostate cancer cases with Gleason score ≧8 compared with that of Gleason score 3+3 in cancer adjacent benign areas (p<0.05, FIG. 19 benign area), but not significantly different in cancer areas, although there is a trend of slight increase

The measurement of the biomarkers in the cancer area and/or non-cancer (benign) areas of the biopsy sample can also be used to predict biochemical recurrence (PSA), which may be used to plan patient treatment.

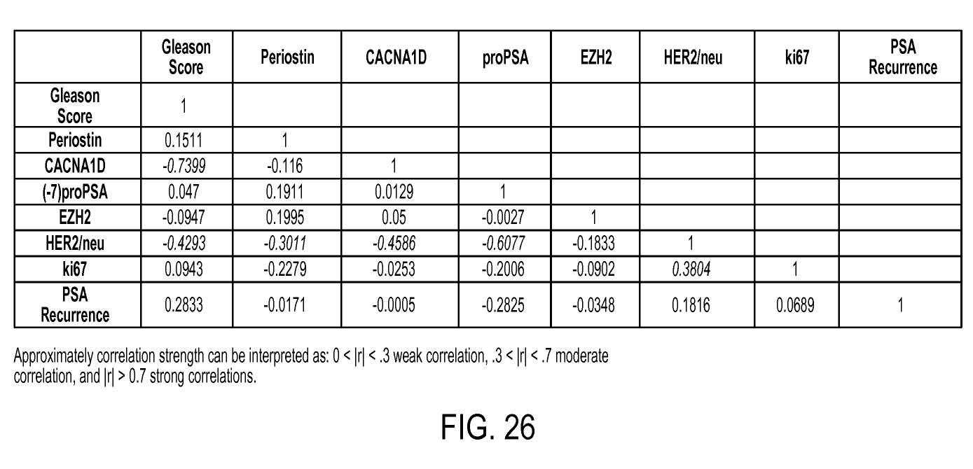

The correlation analysis of the six biomarkers in the cancerous area of the tissue samples evaluated are shown in the graph of FIG. 26 where the correlation strength can be interpreted as: 0<|r|<0.3 weak correlation, 0.3<|r|<0.7 moderate correlation, and |r|>0.7 strong correlations.

Pearson Correlation of Biomarkers with PSA Recurrence and Gleason Scores in TMA Cancer Area.

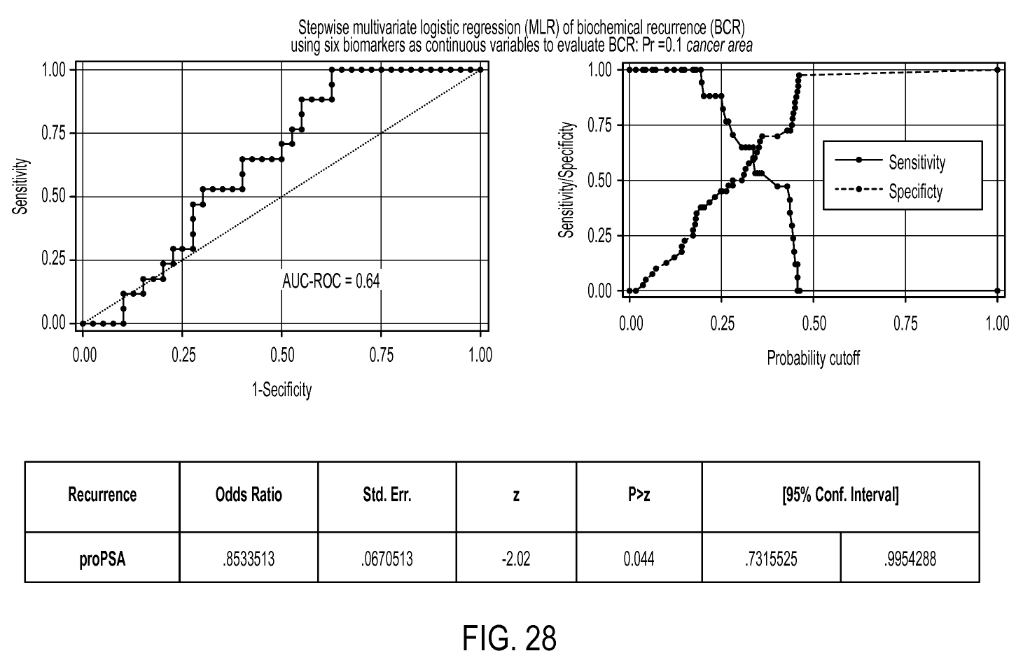

FIG. 27 shows the logistic regression for assessing biochemical recurrence (BCR) as a binary outcome using only Gleason scores as a categorical value to predict BCR in the cancer area with an AUC value of 0.71. Then, FIG. 28 shows the stepwise multivariable logistic regression (MLR) of biochemical recurrence (BCR) using only six biomarkers as continuous variables to evaluate BCR in the cancer area with an AUC value of 0.64.

In FIG. 29, the stepwise multivariable logistic regression (MLR) of biochemical recurrence (BCR) using Gleason grade and the six biomarkers BCR in the cancer area are shown with an AUC value of 0.79 (sensitivity 76.5% and specificity 73.7%). The probability cut of is 32.6%. Combining the measurement of biomarkers in the cancer area with the Gleason score increased the accuracy of predicting biochemical recurrence, wherein the AUC value increased from 0.71 for Gleason score only and 0.64 for biomarkers only to 0.79 for the combination of Gleason score and measurement of biomarkers. Thus, in certain embodiments biopsy samples obtained to monitor cancer recurrence are also analyzed in the cancer areas for at least one of the biomarkers, and up to six, to increase the accuracy for predicting recurrence of prostate cancer.

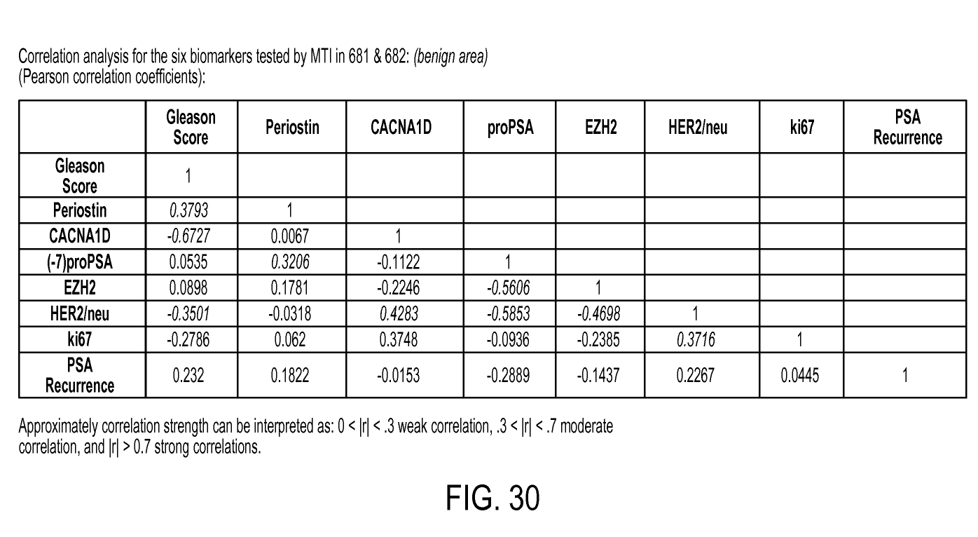

FIGS. 30 through 33 show the same data above for the benign area.

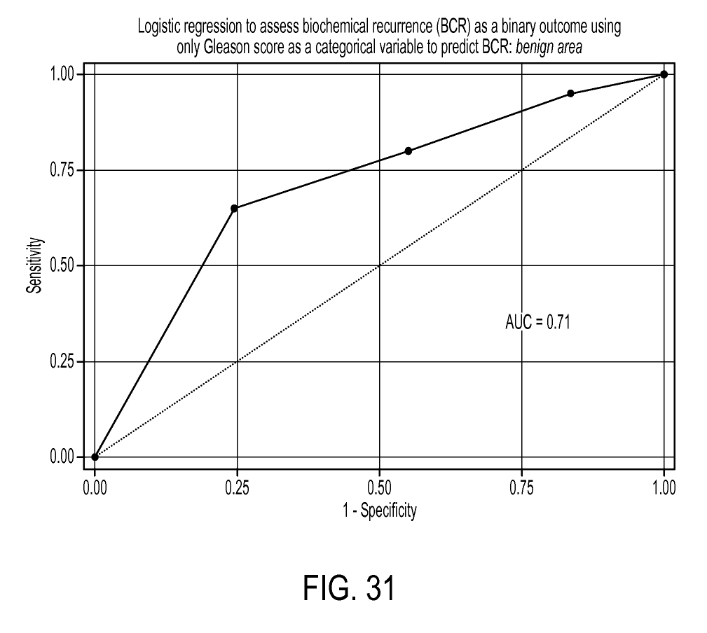

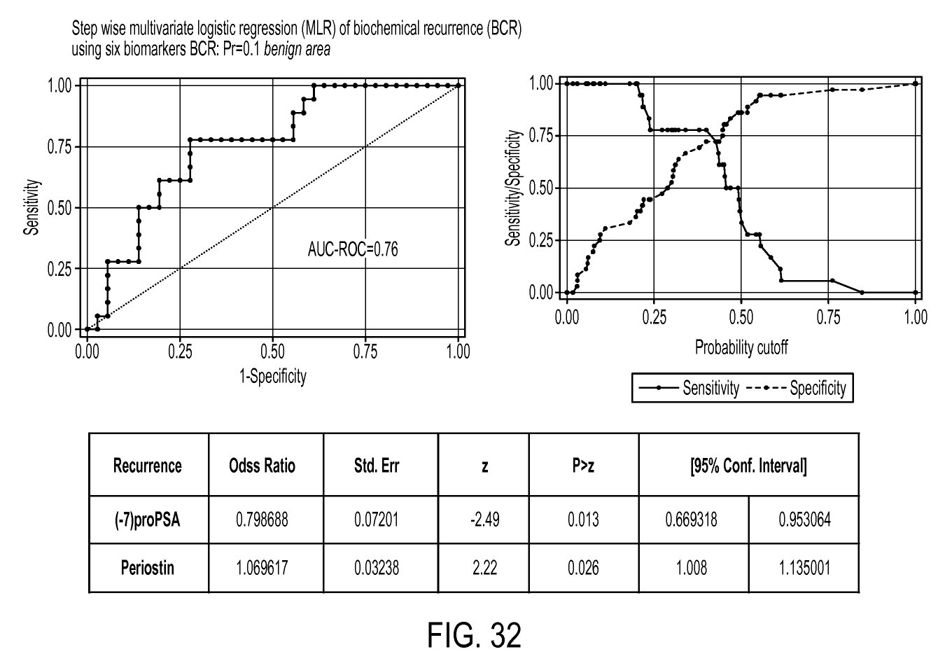

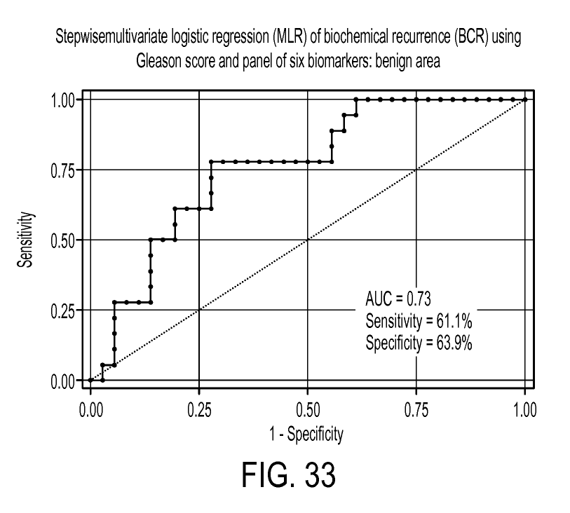

FIG. 31 shows the logistic regression for assessing biochemical recurrence (BCR) as a binary outcome using only Gleason scores as a categorical value to predict BCR in the cancer adjacent benign area with an AUC value of 0.71. FIG. 32 shows the stepwise multivariable logistic regression (MLR) of biochemical recurrence (BCR) using only the six biomarkers as continuous variables to evaluate BCR in the cancer adjacent benign areas with an AUC value of 0.76. In FIG. 33, the stepwise multivariable logistic regression (MLR) of biochemical recurrence (BCR) using Gleason grade and the six biomarkers BCR in the cancer adjacent benign area are shown with an AUC value of 0.73 (sensitivity 61.1% and specificity 63.9%). The probability cut of is 35.3%. In this instance, measuring only the six biomarkers in the cancer adjacent benign areas is a better predictor for recurrence than Gleason score alone or the combination of the Gleason score and measuring the biomarkers. Thus, in certain embodiments biopsy samples obtained to monitor cancer recurrence are analyzed in the cancer adjacent benign areas for at least one of the biomarkers, and up to six, to increase the accuracy for predicting recurrence of prostate cancer as compared to only the Gleason score.

### REFERENCES

The following references are incorporated herein by reference in their entirety:
1. Etzioni, R., et al., Overdiagnosis due to prostate-specific antigen screening: lessons from U.S. prostate cancer incidence trends. J Natl Cancer Inst, 2002. 94(13): p. 981-90.
2. Draisma, G., et al., Lead times and overdetection due to prostate-specific antigen screening: estimates from the European Randomized Study of Screening for Prostate Cancer. J Natl Cancer Inst, 2003. 95(12): p. 868-78.
3. Tornblom, M., et al., Lead time associated with screening for prostate cancer. Int J Cancer, 2004. 108(1): p. 122-9.
4. Cooperberg, M. R., et al., The changing face of low-risk prostate cancer: trends in clinical presentation and primary management. J Clin Oncol, 2004. 22(11): p. 2141-9.
5. Epstein, J. I., et al., Pathologic and clinical findings to predict tumor extent of nonpalpable (stage T1c) prostate cancer. JAMA, 1994. 271(5): p. 368-74.
6. Johansson, J. E., et al., Natural history of early, localized prostate cancer. JAMA, 2004.291(22): p. 2713-9.
7. Bill-Axelson, A., et al., Radical prostatectomy versus watchful waiting in early prostate cancer. N Engl J Med, 2005. 352(19): p. 1977-84.
8. Welch, H. G., L. M. Schwartz, and S. Woloshin, Prostate-specific antigen levels in the United States: implications of various definitions for abnormal. J Natl Cancer Inst, 2005. 97(15): p. 1132-7.
9. Choo, R., et al., Feasibility study: watchful waiting for localized low to intermediate grade prostate carcinoma with selective delayed intervention based on prostate specific antigen, histological and/or clinical progression. J Urol, 2002.167(4): p. 1664-9.
10. Tosoian, J. J., et al., Active surveillance program for prostate cancer: an update of the Johns Hopkins experience. J Clin Oncol. 29(16): p. 2185-90.
11. Klotz, L., Active surveillance for prostate cancer: a review. Curr Urol Rep. 11(3): p. 165-71.
12. Bastian, P. J., et al., Characteristics of insignificant clinical T1c prostate tumors. A contemporary analysis. Cancer, 2004.101(9): p. 2001-5.
13. Duffield, A. S., et al., Radical prostatectomy findings in patients in whom active surveillance of prostate cancer fails. J Urol, 2009.182(5): p. 2274-8.
14. Carter, H. B., et al., Prospective evaluation of men with stage T1C adenocarcinoma of the prostate. J Urol, 1997.157(6): p. 2206-9.
15. Carter, H. B., et al., Expectant management of nonpalpable prostate cancer with curative intent: preliminary results. J Urol, 2002.167(3): p. 1231-4.
16. Mohler J, Bahnson R R, Boston B et al. NCCN clinical practice guidelines in oncology: prostate cancer. J Natl Compr CancNetw 2010; 8: 162-200.
17. Sokoll, L. J., et al., A prospective, multicenter, National Cancer Institute Early Detection Research Network study of [−2]proPSA: improving prostate cancer detection and correlating with cancer aggressiveness. Cancer Epidemiol Biomarkers Prev. 19(5): p. 1193-200.
18 Makarov, D. V., et al., Pro-prostate-specific antigen measurements in serum and tissue are associated with treatment necessity among men enrolled in expectant management for prostate cancer. Clin Cancer Res, 2009.15(23): p. 7316-21.
19. Isharwal, S., et al., ProPSA and diagnostic biopsy tissue DNA content combination improves accuracy to predict need for prostate cancer treatment among men enrolled in an active surveillance program. Urology. 77(3): p. 763 e1-6.
20. Makarov, D. V., et al., Using nuclear morphometry to predict the need for treatment among men with low grade, low stage prostate cancer enrolled in a program of expectant management with curative intent. Prostate, 2008.68(2): p. 183-9.

21. Veltri, R. W., et al., Long-term assessment of prostate cancer progression free survival: evaluation of pathological parameters, nuclear shape and molecular biomarkers of pathogenesis. Prostate, 2008.68(16): p. 1806-15.
22. Isharwal, S., et al., p300 (histone acetyltransferase) biomarker predicts prostate cancer biochemical recurrence and correlates with changes in epithelia nuclear size and shape. Prostate, 2008.68(10): p. 1097-104.
23. Veltri, R. W., et al., Quantitative nuclear morphometry, Markovian texture descriptors, and DNA content captured on a CAS-200 Image analysis system, combined with PCNA and HER-2/neu immunohistochemistry for prediction of prostate cancer progression. J Cell Biochem Suppl, 1994. 19: p. 249-58.
24. Isharwal, S., et al., Prognostic value of Her-2/neu and DNA index for progression, metastasis and prostate cancer-specific death in men with long-term follow-up after radical prostatectomy. Int J Cancer, 2008. 123(11): p. 2636-43.
25. Zhang H, Li X J, Martin D B, & Aebersold R (2003) Identification and quantification of N-linked glycoproteins using hydrazide chemistry, stable isotope labeling and mass spectrometry. Nat Biotechnol 21(6):660-666,
26. Tian Y, Zhou Y, Elliott S, Aebersold R, & Zhang H (2007) Solid-phase extraction of N-linked glycopeptides. Nat Protocols 2:334-339,
27. Tian Y, Bova G S, & Zhang H (2011) Quantitative glycoproteomic analysis of optimal cutting temperature-embedded frozen tissues identifying glycoproteins associated with aggressive prostate cancer. Anal Chem 83(18):7013-7019, 10.1021/ac200815q
28. Hewitt, S. M., J. Dear, and R. A. Star, Discovery of protein biomarkers for renal diseases. J Am Soc Nephrol, 2004. 15(7): p. 1677-89.
29. Chung, J. Y., et al., Transfer and multiplex immunoblotting of a paraffin embedded tissue. Proteomics, 2006.6(3): p. 767-74.
30. Gill, J. E. and M. M. Jotz, Further observations on the chemistry of pararosaniline-Feulgen staining. Histochemistry, 1976.46(2): p. 147-60.
31. Schulte, E. and D. Wittekind, Standardization of the Feulgen-Schiff technique. Staining characteristics of pure fuchsin dyes; a cytophotometric investigation. Histochemistry, 1989.91(4): p. 321-31.
32. Steyerberg, E., Clinical Prediction Models. 2009, New York: Springer.
33. Van Houwelingen, J. C. and S. Le Cessie, Predictive value of statistical models. Stat Med, 1990.9(11): p. 1303-25.
34. Harrell, F. E., Jr., K. L. Lee, and D. B. Mark, Multivariable prognostic models: issues in developing models, evaluating assumptions and adequacy, and measuring and reducing errors. Stat Med, 1996.15(4): p. 361-87.
35. Pencina, M. J., et al., Evaluating the added predictive ability of a new marker: from area under the ROC curve to reclassification and beyond. Stat Med, 2008.27(2): p. 157-72; discussion 207-12.
36. Steyerberg, E. W., et al., Internal and external validation of predictive models: a simulation study of bias and precision in small samples. J Clin Epidemiol, 2003. 56(5): p. 441-7.

The invention has been described with references to a preferred embodiment. While specific values, relationships, materials and steps have been set forth for purposes of describing concepts of the invention, it will be appreciated by persons skilled in the art that numerous variations and/or modifications may be made to the invention as shown in the specific embodiments without departing from the spirit or scope of the basic concepts and operating principles of the invention as broadly described. It should be recognized that, in the light of the above teachings, those skilled in the art can modify those specifics without departing from the invention taught herein. Having now fully set forth the preferred embodiments and certain modifications of the concept underlying the present invention, various other embodiments as well as certain variations and modifications of the embodiments herein shown and described will obviously occur to those skilled in the art upon becoming familiar with such underlying concept. It is intended to include all such modifications, alternatives and other embodiments insofar as they come within the scope of the appended claims or equivalents thereof. It should be understood, therefore, that the invention may be practiced otherwise than as specifically set forth herein. Consequently, the present embodiments are to be considered in all respects as illustrative and not restrictive.

1. A method for identifying a patient for prostate cancer clinical intervention wherein the patient is currently identified for Active Surveillance of the cancer, comprising:
i) measuring a level of at least one biomarker associated with aggressive prostate cancer in a sample from the patient wherein at least one of the biomarkers is CACNA1D;
ii) calculating a probability of aggressive prostate cancer from said biomarker measurements; and,
iii) identifying and selecting the patient for prostate cancer clinical intervention wherein the probability of aggressive prostate cancer indicates a likelihood that a patient has an early form of aggressive prostate cancer.

2. The method of claim 1, wherein the sample is selected for measuring the at least one biomarker if nuclear morphometric test results show a Gleason score <6, <2 cores containing cancer, and <50% per core involved with cancer.

3. The method of claim 1, wherein the at least one biomarker is further selected from the group consisting of Periostin, Her2/neu, EZH2, (−5,−7) ProPSA, Ki67, P300 and PBOV-1.

4. The method of claim 1, wherein said testing step comprises multiplex tissue imprinting (MTI).

5. The method of claim 1, wherein the number of biomarkers tested is selected from the group consisting of at least 2, 3, 4. 5, or 6 biomarkers.

6. The method of claim 1, further comprising comparing presence of the biomarkers in a cancer section of the tissue sample and a benign section of the tissue sample.

7. The method of claim 7, wherein the difference in the presence of the biomarker in the benign section of the tissue sample indicates the patient should be treated.

8. The method of claim 1, wherein the at least one biomarker associated with aggressive prostate cancer is capable of distinguishing indolent CaP from an early stage aggressive CaP.

9. A method for reducing the number of false negative associated with screening for prostate cancer, comprising:
i) categorizing a patient based on measurement of circulating PSA and tissue morphology of a biopsy sample from the patient into low risk categories;
ii) measuring a level of at least one biomarker associated with aggressive prostate cancer in the biopsy sample from those categorized as low risk wherein at least one of the biomarkers is CACA1ND;
iii) calculating a probability of aggressive prostate cancer from said biomarker measurements; and,
iv) re-categorizing a patient into an Intermediate or High Risk category for prostate cancer when the probability of aggressive prostate indicates a likelihood that a patient has an early form of aggressive prostate cancer, whereby the number of false negatives for prostate cancer is reduced.

10. The method of claim 9, wherein low risk categories are defined by one of the following criteria, alone or in combination, i) PSA measurement of 10 ng/ml or less, ii) a Gleason score of 6 or less, iii) no more than 2 cores containing cancer in biopsy sample, iv) 50% or less of core involved with cancer in biopsy sample, and v) PSA density of less than 0.15.

11. The method of claim 9, wherein the low risk categories comprise Low risk and Very Low risk according to Table 1.

12. The method of claim 9, wherein the re-categorized patient is selected for clinical intervention.

13. The method of claim 12, wherein clinical intervention comprises surgery, chemotherapy, targeted drug therapeutic treatment or a combination thereof.

14. The method of claim 9, further comprising comparing presence of the biomarkers in a cancer section of the tissue sample and a benign section of the tissue sample.

15. The method of claim 14, wherein the difference in the presence of the biomarker in the benign section of the tissue sample indicates the patient should be treated.

16. A method for assessing the likelihood that a patient has an early form of aggressive prostate cancer, comprising:
i) assessing tissue morphology of a biopsy sample from the patient including assigning a Gleason score;
ii) measuring a level of prostate specific antigen (PSA) in a blood sample from the patient;
iii) measuring a level of at least one biomarker associated with aggressive prostate cancer in the biopsy sample from the patient wherein at least one of the biomarkers is CACNA1D;
iv) calculating a probability of aggressive prostate cancer from said PSA measurement, biomarker measurements and tissue morphology of the biopsy sample, whereby the likelihood that a patient has an early form of aggressive prostate cancer is determined.

17. The method of claim 16, wherein the measured PSA 10 ng/ml or less.

18. The method of claim 16, wherein the Gleason score is 6 or less.

19. The method of claim 16, wherein the measured PSA 10 ng/ml or less and the Gleason score is 6 or less.

20. The method of claim 19, further comprising selecting those patients with a calculated probability for the likelihood of having an early form of aggressive prostate cancer for treatment.
